(19) United States
(12) Patent Application Publication
(10) Pub. No.: US 2003/0148469 A1
(43) Pub. Date: Aug. 7, 2003

(54) Combinatorial polyketide libraries produced using a modular PKS gene cluster as scaffold

(76) Inventors: Gary Ashley, Alameda, CA (US); Melanie C. Betlach, Burlingame, CA (US); Mary Betlach, San Francisco, CA (US); Robert McDaniel, Palo Alto, CA (US); Li Tang, Foster City, CA (US)

Correspondence Address: Randolph Ted Apple, Morrison & Foerster LLP, 755 Page Mill Road, Palo Alto, CA, 94304-1018, US

(21) Appl. No.: 10/201,365

(22) Filed: Jul. 22, 2002

Related U.S. Application Data
(63) Continuation of application No. 09/141,908, filed on Aug. 28, 1998, now Pat. No. 6,503,741.
Application No. 09/141,908 is a continuation-in-part of application No. 09/073,538, filed on May 6, 1998, now Pat. No. 6,558,942.

(51) Int. Cl. C12P 19/62; C12P 21/02; C12N 1/21; C07H 21/04; C12N 9/10; C12N 15/74

(52) U.S. Cl. 435/76; 435/69.1; 435/252.3; 435/320.1; 536/23.2; 435/193

(57) ABSTRACT

Combinatorial libraries of polyketides can be obtained by suitable manipulation of a host modular polyketide synthase gene cluster such as that which encodes the PKS for picromycin. The combinatorial library is useful as a source of pharmaceutically active compounds. In addition, novel polyketides and antibiotics are prepared using this method.

### CROSS-REFERENCE TO RELATED APPLICATIONS

This application is a continuation-in-part of U.S. Ser. No. 09/073,538 filed May 6, 1998 which is a continuation-in-part of U.S. Ser. No. 08/846,247 filed Apr. 30, 1997. Priority is claimed under 35 USC § 120. Priority is also claimed under 35 USC 119(e) with respect to U.S. Provisional application 60/076,919 filed Mar. 5, 1998 and U.S. Provisional application 60/087,080 filed May 28, 1998. The disclosures of these applications are incorporated herein by reference.

### REFERENCE TO GOVERNMENT FUNDING

This work was supported in part by a grant from the National Institutes of Health, CA66736. The U.S. government has certain rights in this invention.

### TECHNICAL FIELD

The invention relates to the field of novel polyketides and antibiotics and to methods to prepare them. More particularly, it concerns construction of new polyketides and to libraries of polyketides synthesized by polyketide synthases derived from the picromycin PKS and other enzymes derived from Streptomyces venezuelae.

### BACKGROUND ART

Polyketides represent a large family of diverse compounds ultimately synthesized from 2-carbon units through a series of Claisen-type condensations and subsequent modifications. Members of this group include antibiotics such as tetracyclines, anticancer agents such as daunomycin, and immunosuppressants such as FK506 and rapamycin. Polyketides occur in many types of organisms including fungi and mycelial bacteria, in particular, the actinomycetes.

The polyketides are synthesized in vivo by polyketide synthases (PKS). This group of enzymatically active proteins is considered in a different category from the fatty acid synthases which also catalyze condensation of 2-carbon units to result in, for example, fatty acids and prostaglandins. Two major types of PKS are known which are vastly different in their construction and mode of synthesis. These are commonly referred to as Type I or “modular” and Type II, “aromatic.”

The PKS scaffold that is one subject of the present invention is a member of the group designated Type I or “modular” PKS. In this type, a set of separate active sites exists for each step of carbon chain assembly and modification, but the individual proteins contain a multiplicity of such separate active sites. There may be only one multifunctional protein of this type, such as the “fungal” type required for the biosynthesis of 6-methyl salicylic acid (Beck, J. et al., Eur J Biochem (1990) 192:487-498; Davis, R. et al., Abstracts of Genetics of Industrial Microorganism Meeting, Montreal, Abstract P288 (1994)). More commonly, and in bacterial-derived Type I PKS assemblies, there are several such multifunctional proteins assembled to result in the end product polyketide. (Cortes, J. et al., Nature (1990) 348:176; Donadio, S. et al., Science (1991) 252:675; MacNeil, D. J. et al, Gene (1992) 115:119.)

A number of modular PKS genes have been cloned. U.S. Pat. No. 5,252,474 describes cloning of genes encoding the synthase for avermectin; U.S. Pat. No. 5,098,837 describes the cloning of genes encoding the synthase for spiramycin; European application 791,655 and European application 791,656 describe the genes encoding the synthases for tylosin and platenolide respectively.

The PKS for erythromycin, used as an illustrative system is a modular PKS. Erythromycin was originally isolated from S. erythraeus (since reclassified as Saccharopolyspora erythrea) which was found in a soil sample from the Philippine archipelago. Cloning the genes was described by Donadio, S. et al., Science (1991) 252:675. The particulars have been reviewed by Perun, T. J. in Drug Action and Drug Resistance in Bacteria, Vol. 1, S. Mitsuhashi (ed.) University Park Press, Baltimore, 1977. The antibiotic occurs in various glycosylated forms, designated A, B and C during various stages of fermentation. The entire erythromycin biosynthetic gene cluster from S. erythraeus has been mapped and sequenced by Donadio et al. in Industrial Microorganisms: Basic and Applied Molecular Genetics (1993) R. H. Baltz, G. D. Hegeman, and P. L. Skatrud (eds.) (Amer Soc Microbiol) and the entire PKS is an assembly of three such multifunctional proteins usually designated DEBS-1, DEBS-2, and DEBS-3, encoded by three separate genes.

Expression of the genes encoding the PKS complex may not be sufficient to permit the production by the synthase enzymes of polyketides when the genes are transformed into host cells that do not have the required auxiliary phosphopantetheinyl transferase enzymes which posttranslationally modify the ACP domains of the PKS. Genes encoding some of these transferases are described in WO97/13845. In addition, enzymes that mediate glycosylation of the polyketides synthesized are described in WO97/23630. U.S. Ser. No. 08/989,332 filed Dec. 11, 1997 describes the production of polyketides in hosts that normally do not produce them by supplying appropriate phosphopantetheinyl transferase expression systems. The contents of this application are incorporated herein by reference.

There have been attempts to alter the polyketide synthase pathway of modular PKS clusters. For example, European application 238,323 describes a process for enhancing production of polyketides by introducing a rate-limiting synthase gene and U.S. Pat. No. 5,514,544 describes use of an activator protein for the synthase in order to enhance production. U.S. Pat. Nos. 4,874,748 and 5,149,639 describe shuttle vectors that are useful in cloning modular PKS genes in general. Methods of introducing an altered gene into a microorganism chromosome are described in WO93/13663. Modification of the loading module for the DEBS-1 protein of the erythromycin-producing polyketide synthase to substitute the loading module for the avermectin-producing polyketide synthase in order to vary the starter unit was described by Marsden, Andrew F. A. et al. Science (1998) 279:199-202 and Oliynyk, M. et al Chemistry and Biology (1996) 3:833-839. WO 98/01571, published Jan. 15, 1998, describes manipulation of the erythromycin PKS and novel polyketides resulting from such manipulation. In addition, WO 98/0156, also published Jan. 15, 1998 describes a hybrid modular PKS gene for varying the nature of the starter and extender units to synthesize novel polyketides.

In addition, U.S. Pat. Nos. 5,063,155 and 5,168,052 describe preparation of novel antibiotics using modular PKS systems.

Type II PKS, in contrast to modular PKS, include several proteins, each of which is simpler than those found in Type I polyketide synthases. The active sites in these enzymes are used iteratively so that the proteins themselves are generally monofunctional or bifunctional. For example, the aromatic PKS complexes derived from Streptomyces have so far been found to contain three proteins encoded in three open reading frames. One protein provides ketosynthase (KS) and acyltransferase (AT) activities, a second provides a chain length determining factor (CLDF) and a third is an acyl carrier protein (ACP).

The present invention is concerned with PKS systems derived from the modular PKS gene clusters which results in the production of narbomycin in Streptomyces narbonensis and of picromycin in S. venezuelae. Glycosylation of the C5 hydroxyl group of the polyketide precursor, narbonolide, is achieved through an endogenous desosamino transferase. In S. venezuelae, narbomycin is then converted to picromycin by the endogenously produced narbomycin hydroxylase. Thus, as in the case of other macrolide antibiotics, the macrolide product of the PKS is further modified by hydroxylation and glycosylation. The nature of these clusters and their manipulation are further described below.

### DISCLOSURE OF THE INVENTION

The invention provides recombinant materials for the production of libraries of polyketides wherein the polyketide members of the library are synthesized by PKS systems derived from picromycin by using this system as a scaffold or by inserting portions of the picromycin PKS into other PKS scaffolds, and by providing recombinant forms of enzymes that further modify the resulting macrolides. Further, recombinant hosts that are modified to provide only certain activities involved in producing the endogenous antibiotic are described. Generally, many members of these libraries may themselves be novel compounds, and the invention further includes novel polyketide members of these libraries. The invention methods may thus be directed to the preparation of an individual polyketide. The individual polyketide may or may not be novel; in any case the invention provides a more convenient method of preparing it. The resulting polyketides may be further modified to convert them to antibiotics, typically through hydroxylation and/or glycosylation. Modified macrolides that are useful intermediates in the preparation of synthetic antibiotics are of particular interest. The invention also includes methods to recover novel polyketides with desired binding activities by screening the libraries of the invention.

The invention provides for the first time, the complete PKS gene cluster which ultimately results, in S. venezuelae, in the production of picromycin. The ketolide product of this PKS is narbonolide which is glycosylated to obtain narbomycin and then hydroxylated at C12 to obtain picromycin. The enzymes responsible for the glycosylation and hydroxylation are also provided.

Thus, in one aspect, the invention is directed to recombinant materials useful in the production of ketolides and their corresponding antibiotics which contain nucleotide sequences encoding at least one activity, or at least one module, or at least one protein encoded by an open reading frame of the picromycin PKS. The invention is directed also to recombinant materials useful for conversion of ketolides to antibiotics which comprise nucleotide sequences encoding the 12-hydroxylase (the picK gene) and the glycosylation enzyme which provides a glycoside residue at position 5 which enzyme is present in S. narbonensis and S. venezuelae. This aspect also provides methods to obtain the corresponding proteins, ketolides and antibiotics.

These materials are also useful as scaffolds and auxiliary reagents in preparing individual polyketides and combinatorial libraries thereof.

Thus, in another aspect, the invention is directed to a method to prepare a nucleic acid which contains a nucleotide sequence encoding a modified polyketide synthase which method comprises using the picromycin PKS encoding sequence as a scaffold and modifying the portions of the nucleotide sequence that encode enzymatic activities, either by mutagenesis, inactivation, or replacement. The thus modified picromycin PKS encoding nucleotide sequence can then be used to modify a suitable host cell and the cell thus modified employed to produce a polyketide different from that produced by the picromycin PKS. In addition, portions of the picromycin PKS can be inserted into other host scaffolds to modify the products thereof. Portions of the picromycin PKS can be hybridized to portions of other PKS-encoding nucleotide sequences to obtain novel nucleotide sequences with one or more reading frames encoding additional PKS alternatives. The picromycin PKS can itself be manipulated, for example, by fusing two or more of its open reading frames in order to make more efficient the production of the intended macrolide.

In another aspect, the invention relates to conversions effected by the product of the picK gene and by the product of the gene encoding glycosylation enzymes for narbonilide. The invention is also directed to polyketides thus produced and the antibiotics to which they may then be converted.

In another aspect, the invention is directed to a multiplicity of cell colonies comprising a library of colonies wherein each colony of the library contains an expression vector for the production of a different modular PKS, but derived from picromycin PKS. By “derived from” picromycin PKS means simply that at least a portion of the modular PKS is identical to that found in the PKS which results the production of narbonolide and is recognizable as such. The derived portion may, of course, be prepared synthetically as well as prepared directly from DNA that originates in organisms which natively produce narbonolide. In a preferred embodiment, PKS derived from the picromycin PKS is used as a scaffold. The library of different modular PKS is in this case obtained by modifying one or more of the regions of the picromycin PKS gene cluster encoding an enzymatic activity so as to alter that activity, leaving intact the scaffold portions of picromycin PKS gene. If desired, an additional scaffold source may be used creating a hybrid scaffold. In another aspect, the invention is directed to a multiplicity of cell colonies comprising a library of colonies wherein each colony of the library contains a different modular PKS derived from the PKS gene clusters as described above. The invention is also directed to methods to produce libraries of PKS complexes and to produce libraries of polyketides and their corresponding antibiotics by culturing these colonies, as well as to the polyketide and antibiotic libraries so produced. In addition, the invention is directed to methods to screen the resulting polyketide and antibiotic libraries and to novel polyketides and antibiotics contained therein.

### BRIEF DESCRIPTION OF THE DRAWINGS

FIG. 1A is a diagram of the erythromycin PKS complex from S. erythraeus showing the function of each multifunctional protein, and also shows the structure of the ketolide product, 6dEB and of D-desosamine and L-cladinose.

FIG. 1B shows a diagram of the post-PKS biosynthesis of erythromycins A-D.

FIG. 2 is a diagram of DEBS-1 from S. erythraeus showing the functional regions separated by linker regions.

FIG. 3 is a diagram of the picromycin PKS.

FIG. 4 shows the postsynthesis conversion of the ketolide product of the picromycin PKS, narbonolide.

FIG. 5 shows a diagram of the cosmid KOS023-27, a list of the open reading frames contained therein, and the nucleotide sequence and deduced amino acid sequences associated with these reading frames. The nucleotide sequence for the entire cosmid insert is included.

FIG. 6 shows a diagram of the cosmid KOS023-26, a list of the open reading frames contained therein, and the nucleotide sequence and deduced amino acid sequences associated with these reading frames.

### MODES OF CARRYING OUT THE INVENTION

It may be helpful to review the nature of the erythromycin PKS complex and the gene cluster that encodes it as a model for modular PKS, in general. To clarify the terminology, the product of the PKS gene cluster is generally termed a ketolide or macrolide and may or may not have antibiotic activity. It is converted to an antibiotic by additional enzymes not considered part of the PKS cluster. These additional enzymes, in general, provide additional hydroxylation and/or glycosylation of the ketolide PKS product.

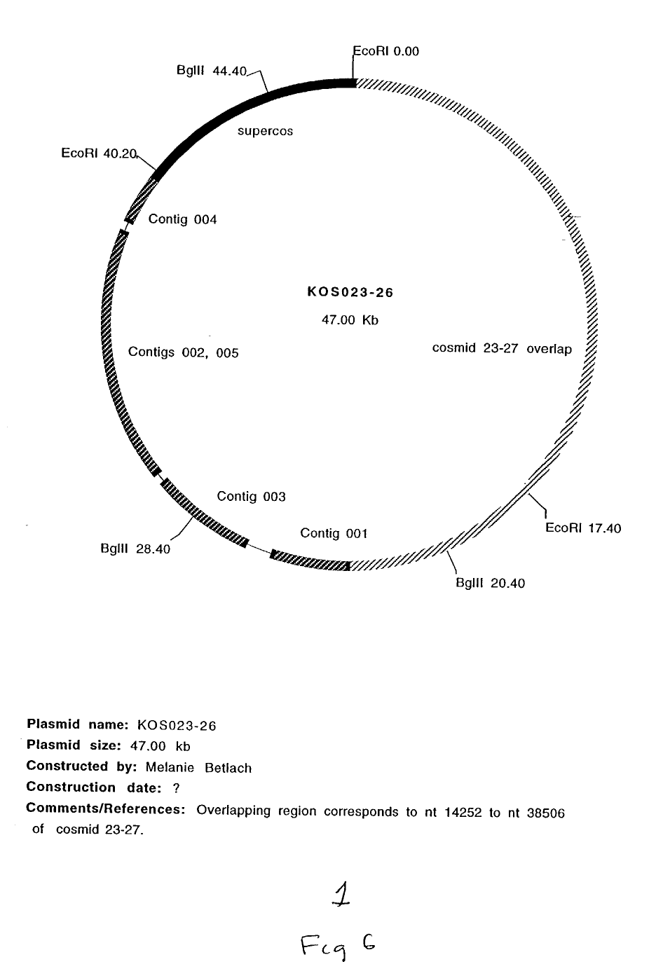

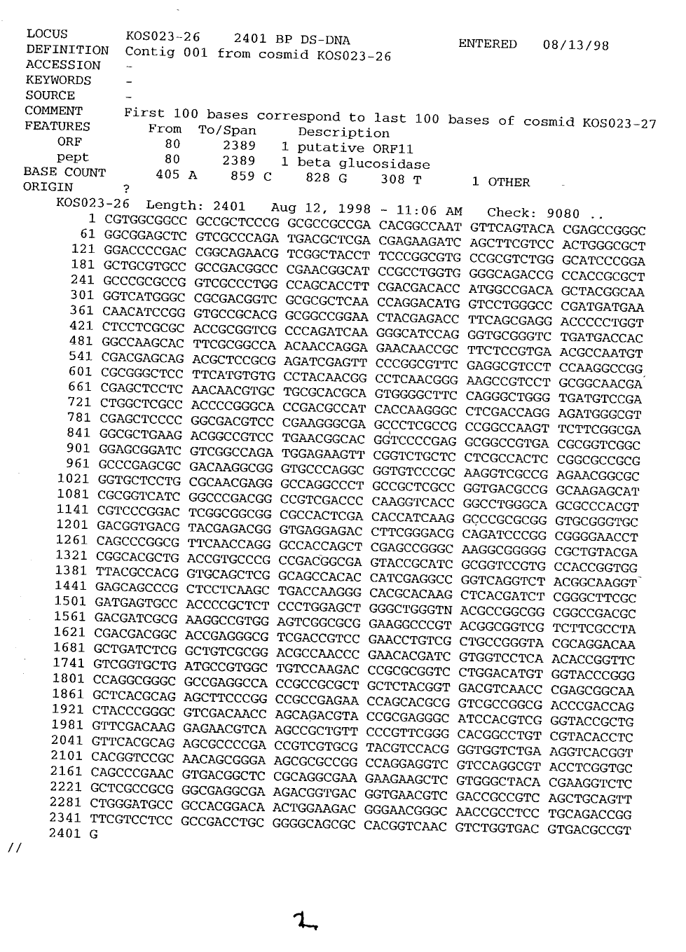

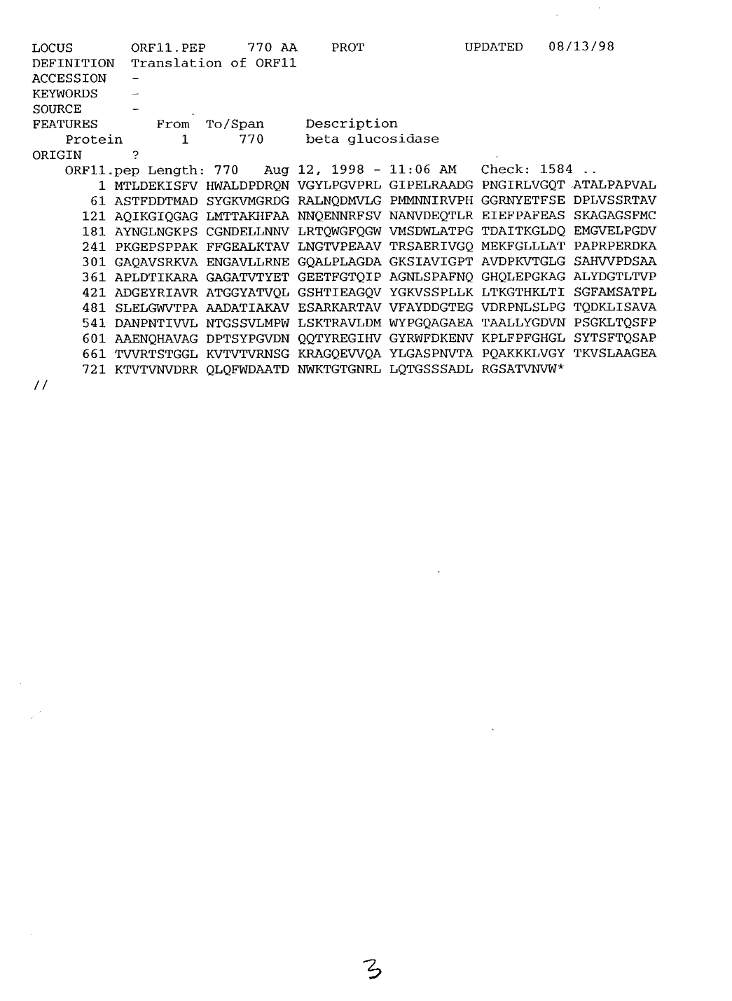

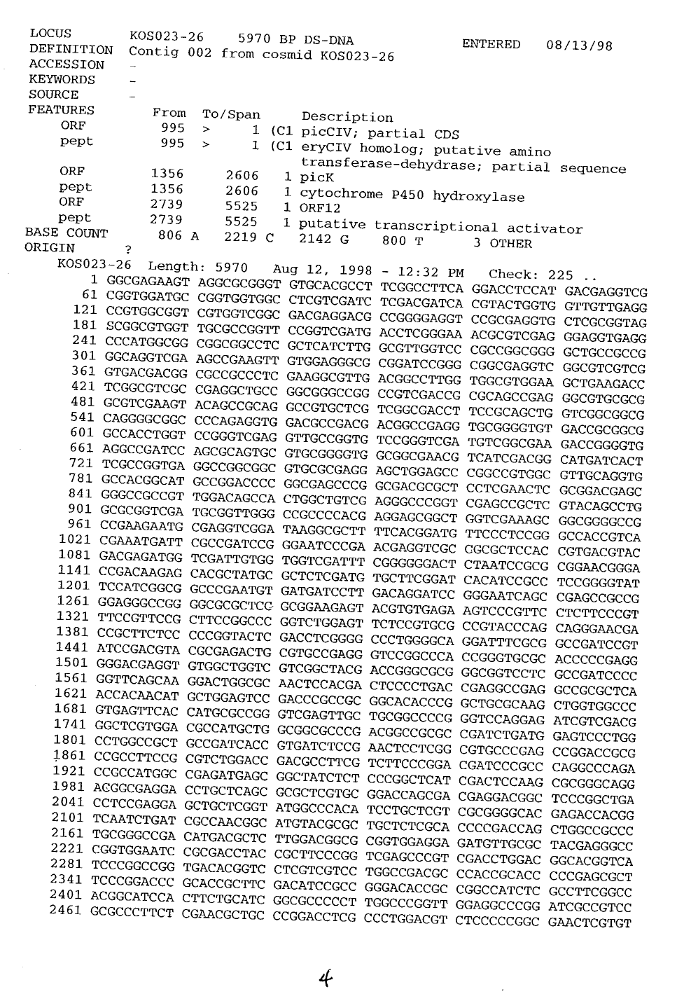

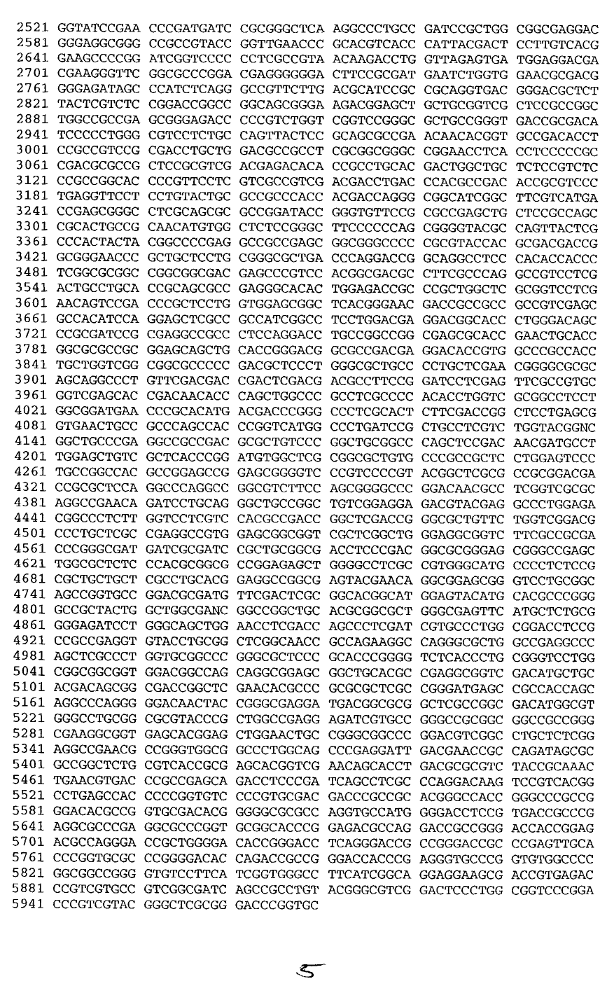

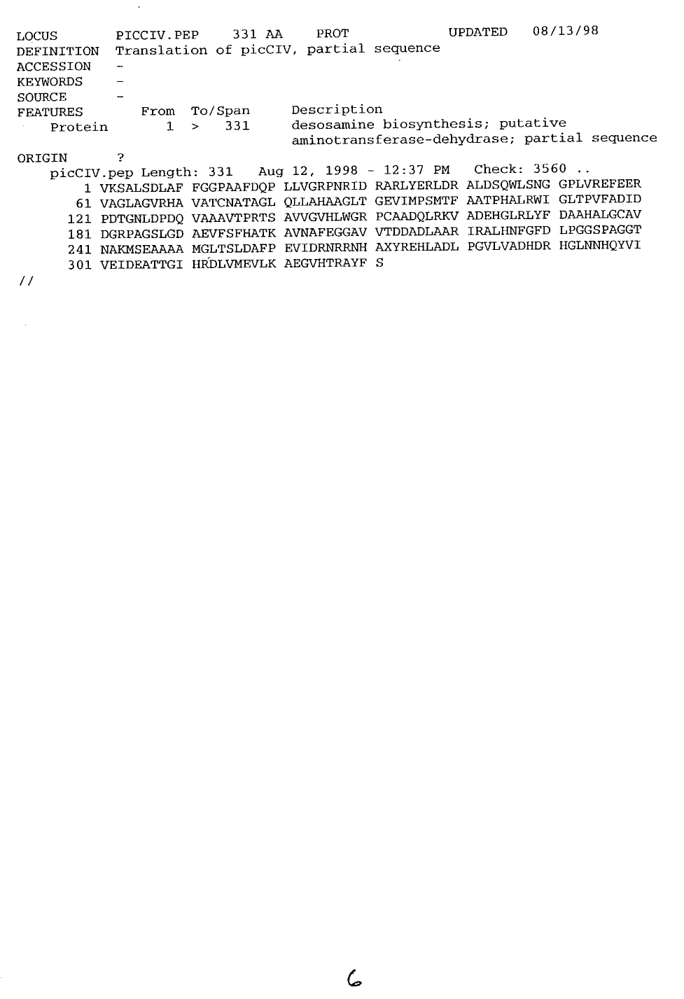

FIG. 1A is a diagrammatic representation of the gene cluster encoding the synthase for the polyketide backbone of the antibiotic erythromycin. The erythromycin PKS protein assembly contains three high-molecular-weight proteins (>200 kD) designated DEBS-1, DEBS-2 and DEBS-3, each encoded by a separate gene (Caffrey et al, FEBS Lett (1992) 304:225). The diagram in FIG. 1A shows that each of the three proteins contains two modules of the synthase—a module being that subset of reactivities required to provide an additional 2-carbon unit to the molecule. As shown in FIG. 1A, modules 1 and 2 reside on DEBS-1; modules 3 and 4 on DEBS-2 and modules 5 and 6 on DEBS-3. The minimal module is typified in module 3 which contains a ketosynthase (KS), an acyltransferase (AT) and an acyl carrier protein (ACP). These three functions are sufficient to activate an extender unit and attach it to the remainder of the growing molecule. Additional activities that may be included in a module relate to reactions other than the Claisen condensation, and include a dehydratase activity (DH), an enoylreductase activity (ER) and a ketoreductase activity (KR). Preceding the first module is a loading domain which contains the AT and ACP activities which catalyze the initial condensation and determine the nature of the starter unit. Although not shown, module 3 has a KR region which has been inactivated (in the native PKS gene cluster) by mutation. The “finishing” of the molecule is regulated by the thioesterase activity (TE) in module 6. This thioesterase appears to catalyze cyclization of the macrolide ring thereby increasing the yield of the polyketide product.

The product in this case is 6dEB; the structure and numbering system for this molecule are shown in FIG. 1A. Conversion to the antibiotics erythromycin A, B, C and D requires two types of reactions, hydroxylation at C-6 and, for erythromycins C and A, at C-12, and glycosylation, generally by D-desosamine or L-mycarose, which may ultimately be converted to cladinose at appropriate locations.

FIG. 1B diagrams the post-PKS biosynthesis of the erythromycins through hydroxylation and addition of glycosyl groups. As shown, 6dEB is converted by the product of the gene eryF to erythronolide B. Erythronolide B (eryB) is hydroxylated at C6. It is believed that this hydroxylation enhances the antibiotic activity. The hydroxylase is not part of the PKS per se; it is nevertheless endogenous to S. erythraeus. Erythronolide B is glycosylated by the product of the eryB gene to obtain 3-O-mycarosylerythronolide B which contains L-mycarose at position 3. This product, 3-O-mycarosylerythronolide B serves as a precursor for all of the erythromycin antibiotics. It is first converted to erythromycin D by the enzyme encoded by eryC by glycosylation with D-desosamine at position 5. Erythromycin D, therefore, differs from 6dEB through glycosylation and by the addition of a hydroxyl group at position 6. Erythromycin D can be converted to erythromycin B in a reaction catalyzed by the product of the eryG gene by methylating the L-mycarose residue at position 3. Erythromycin D is converted to erythromycin C by the addition of a hydroxyl group at position 12. This conversion is catalyzed by a hydroxylase that is the product of the eryK gene. The analogous picK gene is provided by the present invention. Erythromycin A is obtained from erythromycin C by methylation of the mycarose residue catalyzed by the product of the eryG gene. The series of erythromycin antibiotics, then, differs in the level of hydroxylation of the polyketide framework and by the methylation status of the glycosyl residues.

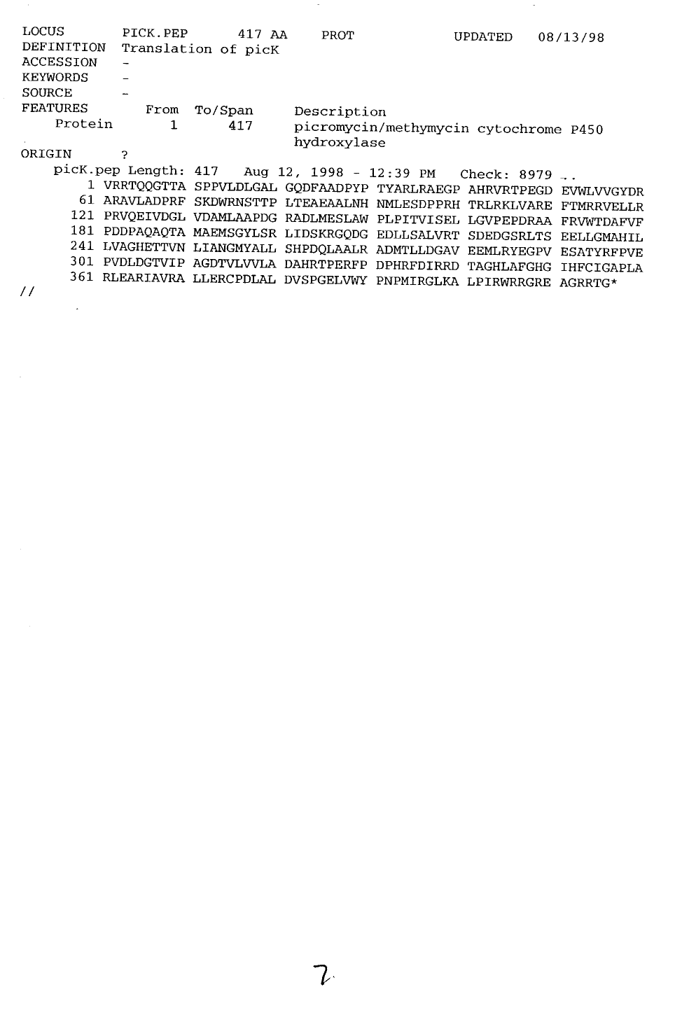

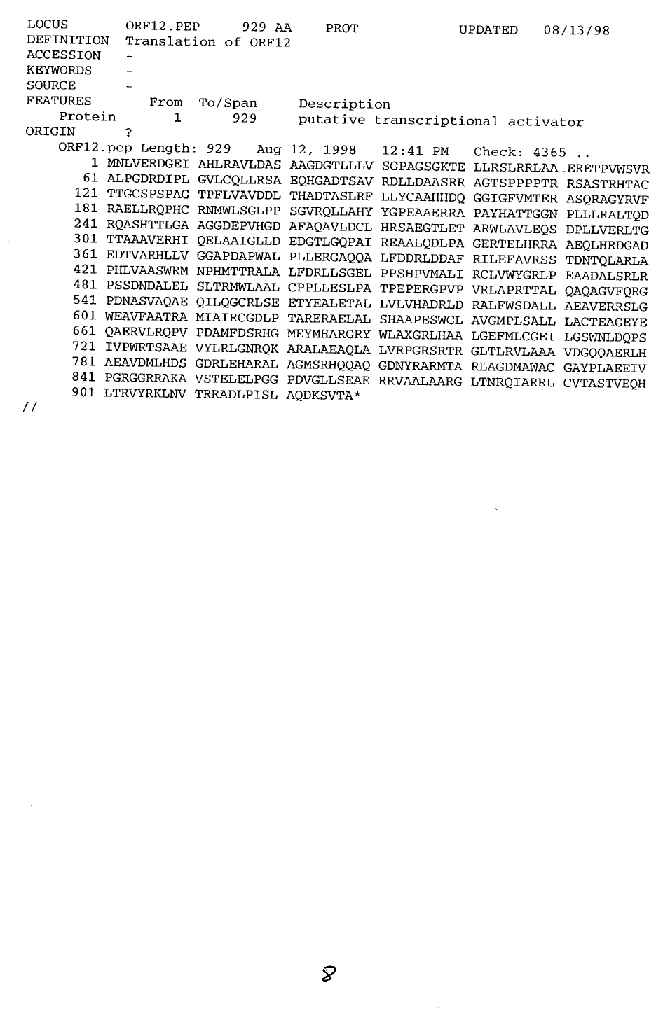

FIG. 2 shows a detailed view of the regions in the first two modules of the erythromycin PKS which comprise the first open reading frame encoding DEBS-1. The regions that encode enzymatic activities are separated by linker or “scaffold”-encoding regions. These scaffold regions encode amino acid sequences that space the enzymatic activities at the appropriate distances and in the correct order. Thus, these linker regions collectively can be considered to encode a scaffold into which the various activities are placed in a particular order and spatial arrangement. This organization is similar in the remaining modules, as well as in other naturally occurring modular PKS gene clusters.

The three DEBS-1, 2 and 3 proteins are encoded by the genetic segments ery-AI, ery-AII and ery-AIII, respectively. These reading frames are located on the bacterial chromosome starting at about 10 kb distant from the erythromycin resistance gene (ermE or eryR).

The detailed description above referring to erythromycin is typical for modular PKS in general. Thus, rather than the illustrated erythromycin, the polyketide synthases making up the libraries of the invention can be derived from the synthases of other modular PKS, such as those which result in the production of rapamycin, avermectin, FK-506, FR-008, monensin, rifamycin, soraphen-A, spinocyn, squalestatin, or tylosin, and the like.

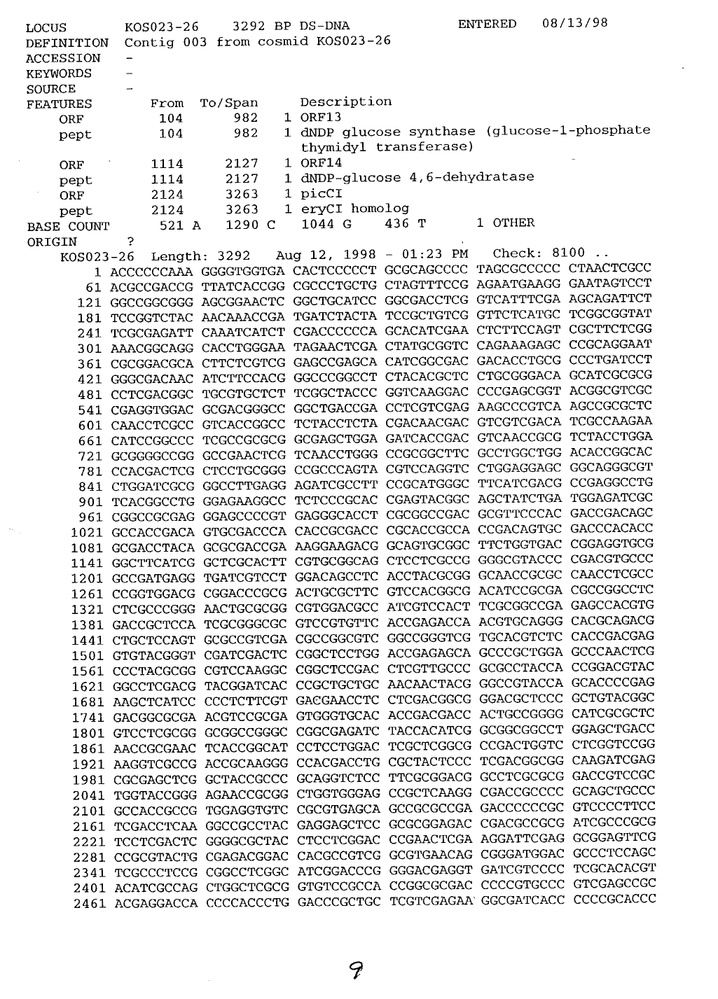

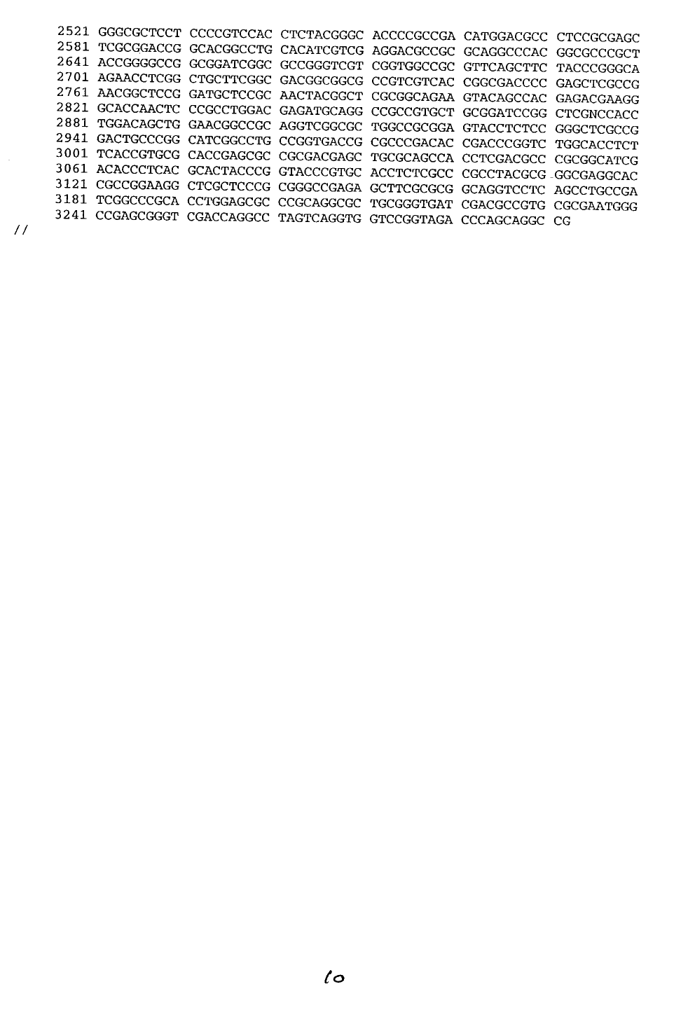

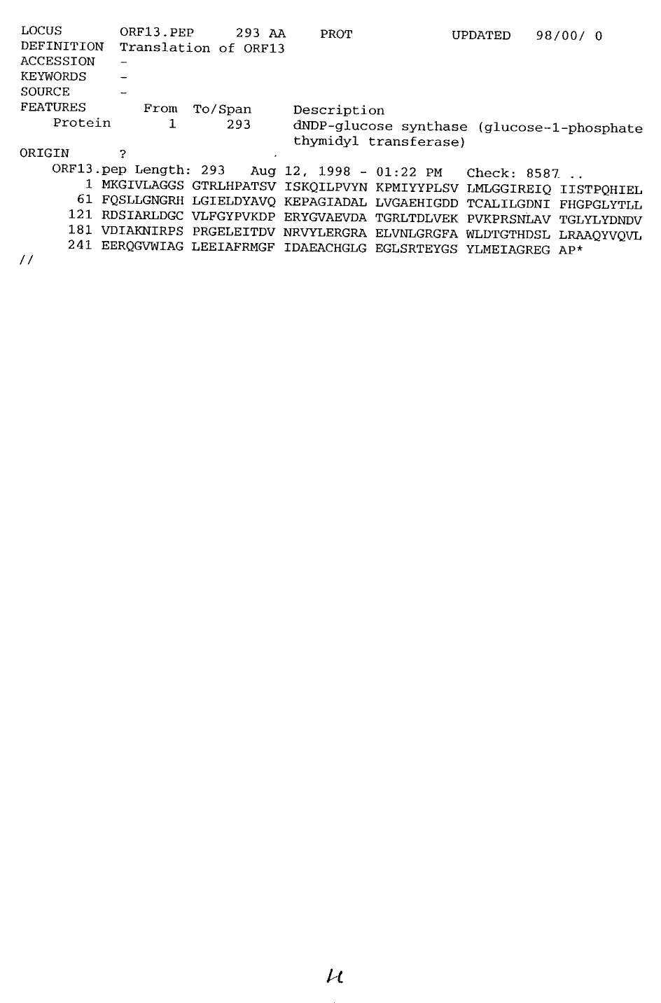

A focus of the present invention is the provision of the nucleotide sequences of the picromycin PKS as well as the nucleotide sequences of genes encoding enzymes which catalyze the further modification of the ketolides produced by the picromycin PKS. FIG. 3 shows a diagram of the picromycin PKS provided by the invention. As compared to the erythromycin PKS, there are many similarities. Both encode enzymes that result in 14-member macrolides; therefore, each contains six modules. The six modules of the picromycin PKS, however, reside on four, rather than three reading frames; modules 5 and 6 are encoded on separate reading frames. As shown in FIG. 3, the activities associated with each module of the picromycin PKS are similar to erythromycin, but there are some important differences.

The loading domain of the picromycin PKS, unlike that of erythromycin, contains an inactivated ketosynthase (KS) domain. Sequence analysis indicates that this domain is enzymatically inactivated as a critical cysteine residue in the motif TVDACSSSL, which is highly conserved among KS domains, is replaced by a glutamine. Such inactivated KS domains are also found in the 16-membered macrolides carbomycin, spiromycin, tyrosin and nidamycin. Thus, in effect, the loading domains of the picromycin and erythromycin PKS appear functionally similar. Modules 1, 3, 4, and 6 are also functionally similar. In both cases, module 3 contains a ketoreductase-encoding region which is inactive. The major functional differences between the two PKS nucleotide sequences occur in modules 2 and 5. This results in structural differences in the resulting ketolides at carbons 10, 11 (module 2) and carbon 3 (module 5). The acyl transferase in module 2 of the picromycin PKS is specific for malonyl CoA, rather than methylmalonyl CoA and thus results in the lack of a methyl group at position 10. Further, the presence of a dehydrase (DH) activity in module 2 results in a double bond between carbons 10 and 11; the ketoreductase present in module 2 in the erythromycin PKS results in a hydroxyl group at position 11.

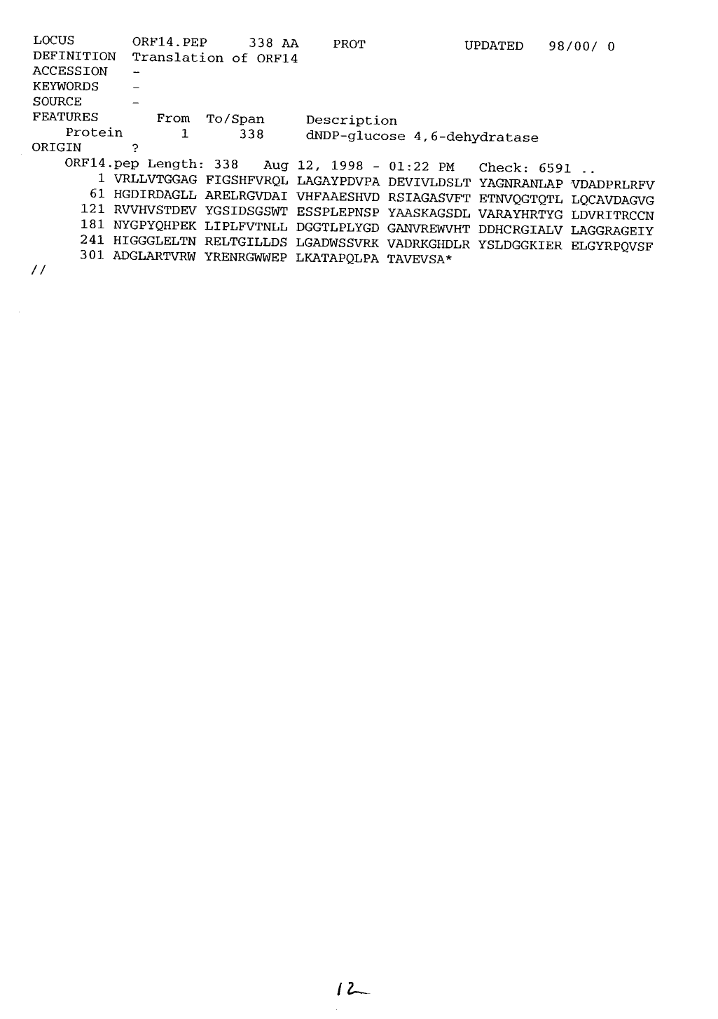

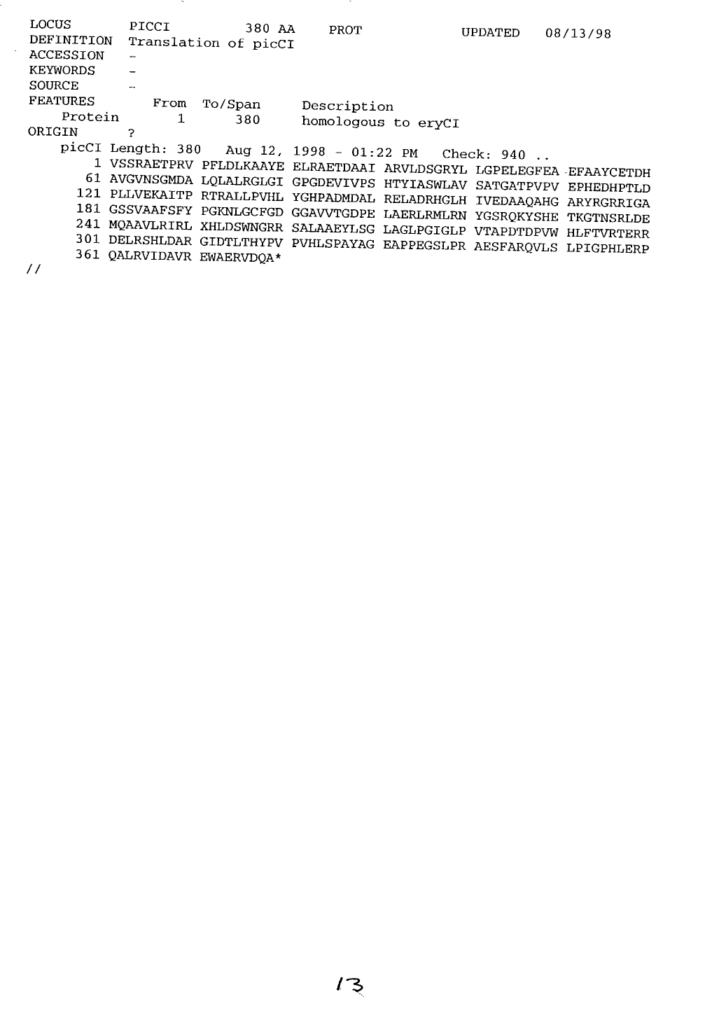

Like erythromycin, picromycin itself results from further modifications catalyzed by enzymes not part of the PKS. This series of reactions is shown in FIG. 4. As shown, the product ketolide, narbonolide, is converted to narbomycin by glycosylation with desosamine and then hydroxylated at the 12-position by the product of the picK gene.

The present invention provides all of the necessary nucleotide sequences for manipulating the picromycin PKS as well as the postmacrolide synthesis enzymes. These materials are contained on pKOS023-27 and pKOS023-26, both deposited at the ATCC under the terms of the Budapest Convention on August __, 1998, and provided accession numbers ATCC ______ and ATCC ______, respectively.

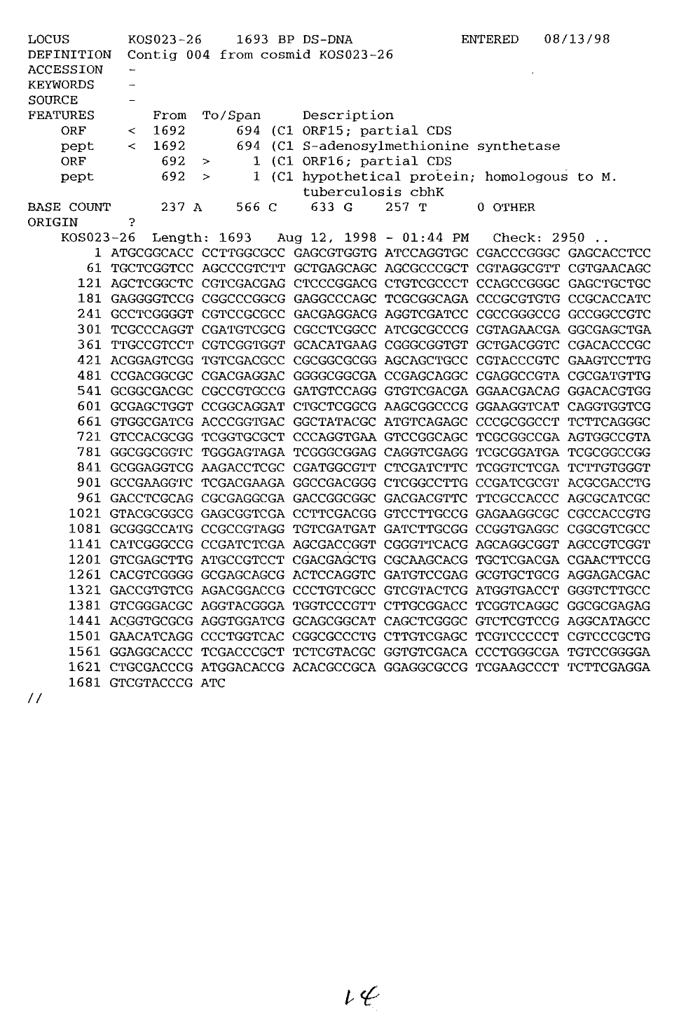

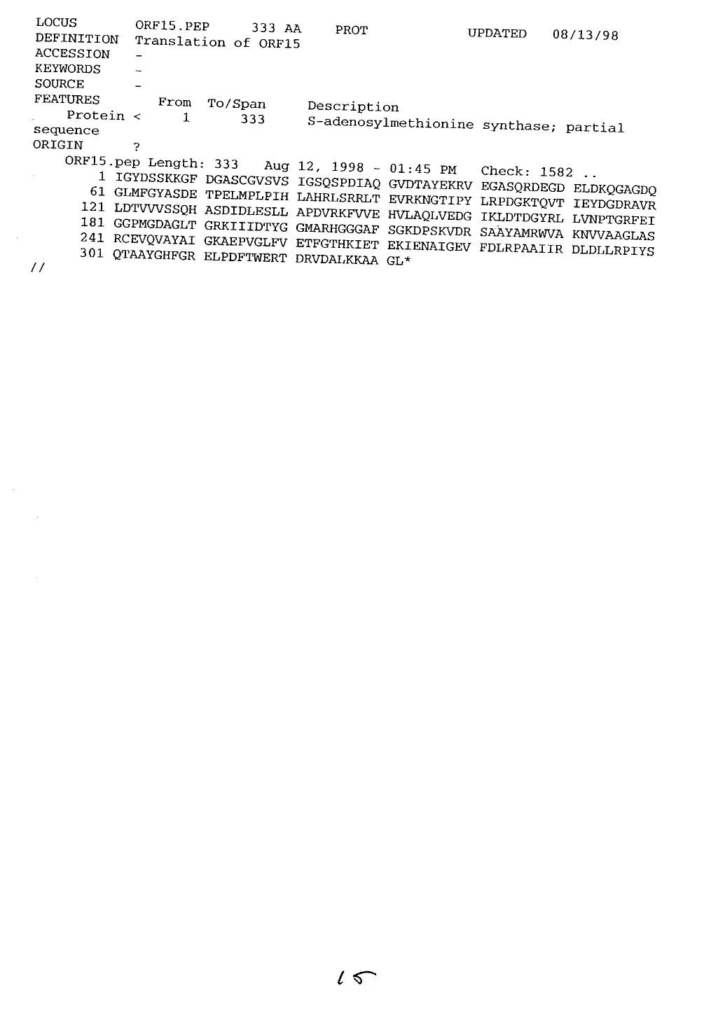

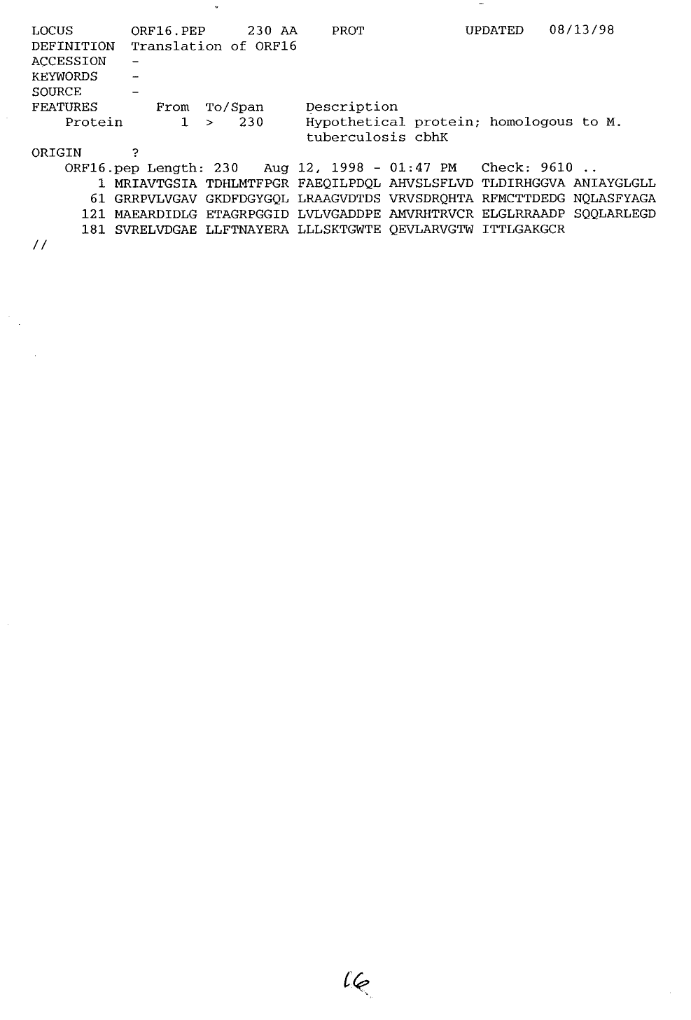

FIG. 5 shows a diagram of pKOS023-27 which contains the entire picromycin PKS along with three additional open reading frames at the C-terminus. The gene product of ORF1 shows a high degree of similarity to all of the non-PKS thioesterases; with an identity of 51%, 49%, 45% and 40% as compared to those of Amycolatopsis mediterranae, S. griseus, S. fradiae and Saccharopolyspora erythreae, respectively. The product of ORF2 shows 48% identity to the dnrQ gene product of S. peucetius. The product of ORF2 is the desosamino transferase which converts narbonolide to narbonomycin. The product of ORF3 also has 50% identity to a glycotransferase.

FIG. 5 also provides the complete nucleotide sequence of pKOS023-27 on pages 3-14 thereof. Pages 15-23 contain the deduced amino acid sequences of the four open reading frames of the PKS and the additional open reading frames at the C-terminus.

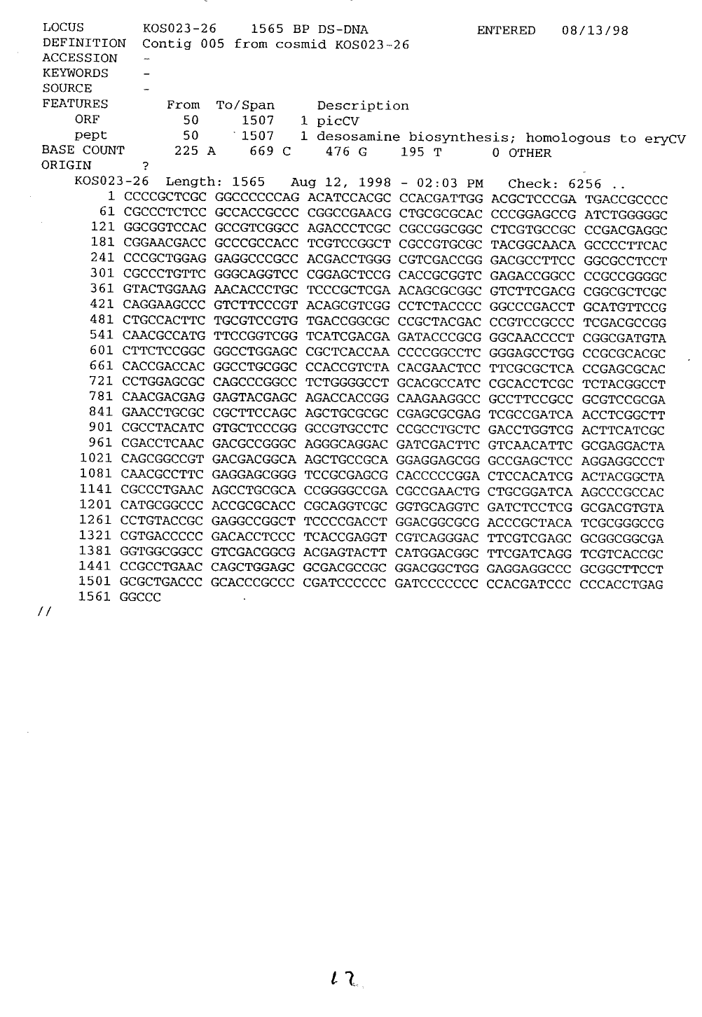

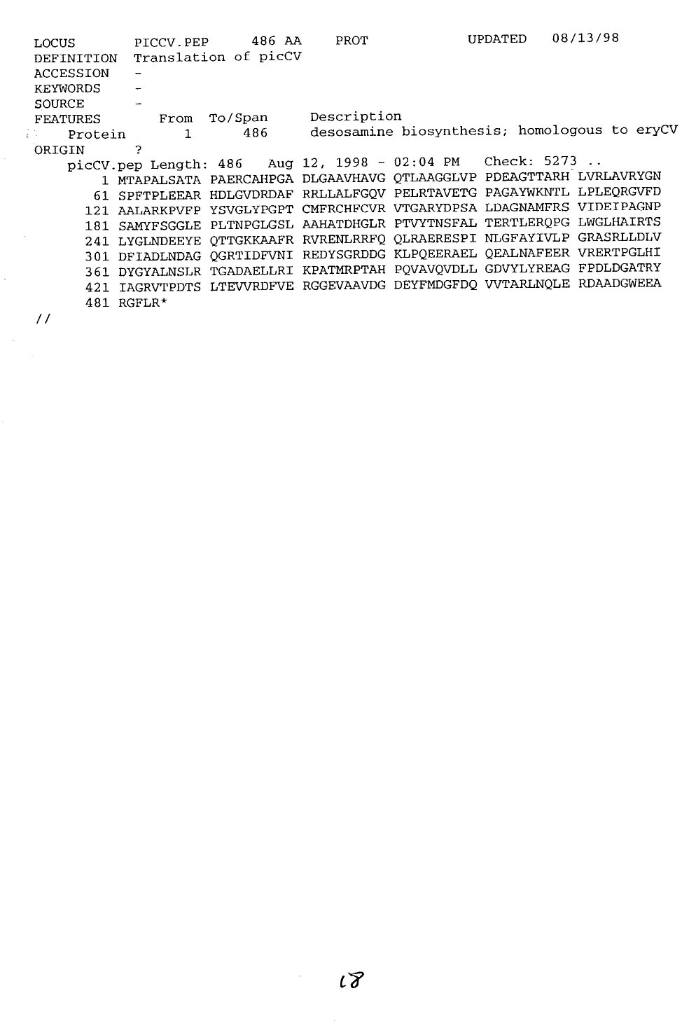

FIG. 6 shows the structure of pKOS023-26 which contains a region of overlap with pKOS023-27 representing nucleotides 14252 to nucleotides 38506 of pKOS023-27. The nucleotide sequences of five contigs contained in pKOS023-26 are provided in FIG. 6 along with the translations of open reading frames contained therein. Pages 2-3 show contig 1 and a translation of the reading frame contained therein; pages 4-8 provide the corresponding information for contig 2; pages 9-13 for contig 3; pages 14-16 for contig 4; and pages 17-18 for contig 5. These open reading frames have been assigned as follows:

In contig 001, one reading frame, ORF11 encodes a glucosidase.

In contig 2, the three reading frames include a reading frame encoding a 3,4-dehydratase designated picC11V which is a homolog of eryCIV. A second reading frame is the picK gene which is a cytochrome p450 hydroxylase responsible for hydroxylating C12 of glycosylated narbomycin. The third reading frame designated ORF12 is putatively a regulatory gene.

In contig 003, one reading frame, designated ORF13 is an NDP glucose synthase and a second gene, designated ORF14 encodes an NDP glucose 4,6-dehydratase. The third open reading frame has been designated picC1 as it appears to be homologous to the eryC1 gene.

In contig 004, the two open reading frames are ORF15 which encodes an S-adenosyl methionine synthase and ORF16 which is a homolog of the M. tuberculosis cbhK gene. Contig 5 contains one reading frame which is designated picCV, a homolog to the eryCV gene which encodes a protein that catalyzes desosamine synthesis.

Thus, nucleotide sequences encoding the entire picromycin PKS have been provided, along with those encoding the enzymes for essential further modification of the resulting ketolide. picK is included in pKOS023-26 contig 002 and the gene encoding the glycosylation enzyme for conversion of narbonolide to narbomycin is shown as ORF2 in FIG. 5.

The availability of these nucleotide sequences permits their use in recombinant procedures for production of desired portions of the picromycin PKS and for production of the proteins useful in postmacrolide conversions. A portion of the PKS which encodes a particular activity can be isolated and manipulated, for example, by replacing the corresponding region in a different modular PKS. In addition, individual modules of the PKS may be ligated into suitable expression systems and used to produce the encoded portion of the protein encoded by the open reading frame which may be isolated and purified, or which may be employed in situ to effect polyketide synthesis. Depending on the host for the recombinant production of the module or an entire open reading frame, or combination of open reading frames, suitable control sequences such as promoters, termination sequences, enhancers, and the like are ligated to the nucleotide sequence encoding the desired protein. Suitable control sequences for a variety of hosts are well known in the art.

If the hosts ordinarily produce polyketides, it may be desirable to modify them so as to prevent the production of endogenous polyketides by these hosts. Such hosts have been described, for example, in U.S. Pat. No. 5,672,491, incorporated herein by reference. In such hosts, however, it may not be necessary to provide enzymatic activity for posttranslational modification of the enzymes that make up the recombinantly produced polyketide synthase. In particular, these hosts generally contain suitable enzymes, designated holo-ACP synthases, for providing a pantotheinyl residue needed for functionality of the synthase. However, in hosts such as yeasts, plants, or mammalian cells which ordinarily do not produce polyketides, it may be necessary to provide, also typically by recombinant means, suitable holo-ACP synthases to convert the recombinantly produced PKS to functionality. Provision of such enzymes is described, for example, in PCT application WO 98/27203, incorporated herein by reference.

Thus, included within the scope of the invention in addition to isolated nucleic acids containing the desired nucleotide sequences encoding activities, modules or open reading frames of PKS as well as glycosylation and hydroxylation enzymes, are recombinant expression systems containing these nucleotide sequences wherein the encoding nucleotide sequences are operably linked to promoters, enhancers, and/or termination sequences which operate to effect expression of the encoding nucleotide sequence in host cells compatible with these sequences; host cells modified to contain these sequences either as extrachromosomal elements or vectors or integrated into the chromosome, and methods to produce PKS and post-PKS enzymes as well as polyketides and antibiotics using these modified host cells.

The availability of these nucleotide sequences also expands the possibility for the production of novel polyketides and their corresponding antibiotics using host cells modified to contain suitable expression systems for the appropriate enzymes. By manipulating the various activity-encoding regions of a donor PKS by replacing them into a scaffold of a different PKS or by forming hybrids instead of or in addition to such replacements or other mutagenizing alterations, a wide variety of polyketides and corresponding antibiotics may be obtained.

The availability of the hydroxylase encoded by the picK gene in recombinant form is of great significance in this regard as the enzyme appears to accept a wide variety of substrates. Thus, additional hydroxylation reactions can be carried out with respect to large numbers of polyketides.

Thus, in addition to the novel polyketides described in parent application U.S. Ser. No. 09/073,538, filed May 6, 1998, the invention includes novel hydroxylated polyketides of the formula

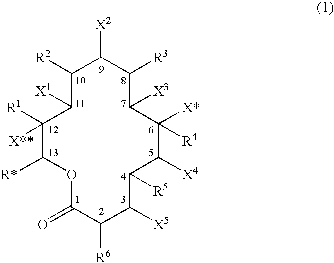

including the glycosylated and isolated stereoisomeric forms thereof,

wherein R* is a straight-chain, branched or cyclic saturated or unsaturated substituted or unsubstituted hydrocarbyl of 1-15C;

each of R¹-R⁶ is independently H or alkyl (1-4C);

each of X¹-X⁵ is independently H₂, HOH or ═O; or

each of X¹-X⁵ is independently H and the compound of formula 5 contains a π-bond in the ring adjacent to the position of said X at 2-3, 4-5, 6-7, 8-9 and/or 10-11; and

wherein at least one of X* and X** is OH; and

wherein at least two of R¹-R⁶ are alkyl.

Hydroxylated forms at the C6 and C12 positions are facilitated by the availability of the relevant hydroxylases. As mentioned above, the C12 hydroxylase encoded by the picK gene is particularly advantageous as it will accept a wide variety of polyketide precursors wherein X** is H.

Hydroxylation can be achieved by a number of approaches. First, the hydroxylation may be performed in vitro using purified hydroxylase or the relevant hydroxylase produced recombinantly from its retrieved gene. Alternatively, hydroxylation may be effected by supplying the nonhydroxylated precursor to a cell which provides the appropriate hydroxylase, either natively, or by virtue of recombinant modification. The availability of the 12-hydroxylase encoded by the picK gene is helpful in providing a cellular environment with the appropriate hydroxylase produced recombinantly. Alternatively, a native source of the hydroxylase, such as S. venezuelae may conveniently be used, either by providing the unhydroxylated ketolide to the cells, or preferably by generating the desired ketolide through recombinant modification of these cells, preferably concomitantly with deleting the ability of the host cell to produce its own polyketide.

The invention provides libraries or individual modified forms, ultimately of polyketides, by generating modifications in the picromycin PKS or other naturally occurring PKS gene cluster so that the protein complexes produced by the cluster have altered activities in one or more respects, and thus produce polyketides other than the natural product of the PKS. Novel polyketides may thus be prepared, or polyketides in general prepared more readily, using this method. By providing a large number of different genes or gene clusters derived from a naturally occurring PKS gene cluster, each of which has been modified in a different way from the native cluster, an effectively combinatorial library of polyketides can be produced as a result of the multiple variations in these activities. As will be further described below, the metes and bounds of this derivation can be described on both the protein level and the encoding nucleotide sequence level.

As described above, a modular PKS “derived from” the picromycin or other naturally occurring PKS includes a modular polyketide synthase (or its corresponding encoding gene(s)) that retains the scaffolding of all of the utilized portion of the naturally occurring gene. (Not all modules need be included in the constructs.) On the constant scaffold, at least one enzymatic activity is mutated, deleted or replaced, so as to alter the activity. Alteration results when these activities are deleted or are replaced by a different version of the activity, or simply mutated in such a way that a polyketide other than the natural product results from these collective activities. This occurs because there has been a resulting alteration of the starter unit and/or extender unit, and/or stereochemistry, and/or chain length or cyclization and/or reductive or dehydration cycle outcome at a corresponding position in the product polyketide. Where a deleted activity is replaced, the origin of the replacement activity may come from a corresponding activity in a different naturally occurring polyketide synthase or from a different region of the picromycin PKS. Any or all of the picA, picB, picC and picD genes (see FIG. 3) may be included in the derivative or portions of any of these may be included; but the scaffolding of the resulting PKS protein is retained in whatever derivative is considered.

The derivative may contain preferably at least a thioesterase activity from the picromycin or other naturally occurring PKS gene cluster.

In summary, a polyketide synthase “derived from” the picromycin PKS includes those which contain the scaffolding encoded by all or the portion employed of the picromycin synthase gene, contains at least two modules that are functional, preferably three modules, and more preferably four or more modules and contains mutations, deletions, or replacements of one or more of the activities of these functional modules so that the nature of the resulting polyketide is altered. This definition applies both at the protein and genetic levels. Particular preferred embodiments include those wherein a KS, AT, KR, DH or ER has been deleted or replaced by a version of the activity from a different PKS or from another location within the same PKS. Also preferred are derivatives where at least one noncondensation cycle enzymatic activity (KR, DH or ER) has been deleted or wherein any of these activities has been mutated so as to change the ultimate polyketide synthesized.

Conversely, also included within the definition of PKS “derived from the picromycin PKS” are functional PKS modules or their encoding genes wherein at least one portion, preferably two portions, of the picromycin activities have been inserted. Exemplary, for example, is the use of the picromycin acyl transferase (AT) for module 2 which accepts a malonyl CoA extender unit rather than methyl malonyl CoA. Other examples include insertion of portions of noncondensation cycle enzymatic activities, or other regions of picromycin synthase activity. Again, the “derived from” definition applies to the PKS at both the genetic and protein levels.

Thus, there are five degrees of freedom for constructing a polyketide synthase in terms of the polyketide that will be produced. First, the polyketide chain length will be determined by the number of modules in the PKS. Second, the nature of the carbon skeleton of the PKS will be determined by the specificities of the acyl transferases which determine the nature of the extender units at each position—e.g., malonyl, methyl malonyl, or ethyl malonyl, etc. Third, the loading domain specificity will also have an effect on the resulting carbon skeleton of the polyketide. Thus, the loading domain may use a different starter unit, such as acetyl, propionyl, butyryl and the like. Fourth, the oxidation state at various positions of the polyketide will be determined by the dehydratase and reductase portions of the modules. This will determine the presence and location of ketone, alcohol, double bonds or single bonds in the polyketide. Finally, the stereochemistry of the resulting polyketide is a function of three aspects of the synthase. The first aspect is related to the AT/KS specificity associated with substituted malonyls as extender units, which affects stereochemistry only when the reductive cycle is missing or when it contains only a ketoreductase since the dehydratase would abolish chirality. Second, the specificity of the ketoreductase will determine the chirality of any β-OH. Finally, the enoyl reductase specificity for substituted malonyls as extender units will influence the result when there is a complete KR/DH/ER available.

In the working examples below, in manipulating the foregoing variables for varying loading domain specificity which controls the starter unit, a useful approach is to modify the KS activity in module 1 which results in the ability to incorporate alternative starter units as well as module 1 extended units. This approach was illustrated in PCT application US/96/11317, incorporated herein by reference, wherein the KS-I activity was inactivated through mutation. Polyketide synthesis is then initiated by feeding chemically synthesized analogs of module 1 diketide products. Working examples of this aspect are also presented hereinbelow.

Thus, the modular PKS systems, and in particular, the picromycin PKS system, permit a wide range of polyketides to be synthesized. As compared to the aromatic PKS systems, a wider range of starter units including aliphatic monomers (acetyl, propionyl, butyryl, isovaleryl, etc.), aromatics (aminohydroxybenzoyl), alicyclics (cyclohexanoyl), and heterocyclics (thiazolyl) are found in various macrocyclic polyketides. Recent studies have shown that modular PKSs have relaxed specificity for their starter units (Kao et al. Science (1994), supra). Modular PKSs also exhibit considerable variety with regard to the choice of extender units in each condensation cycle. The degree of β-ketoreduction following a condensation reaction has also been shown to be altered by genetic manipulation (Donadio et al. Science (1991), supra; Donadio, S. et al. Proc Natl Acad Sci USA (1993) 90:7119-7123). Likewise, the size of the polyketide product can be varied by designing mutants with the appropriate number of modules (Kao, C. M. et al. J Am Chem Soc (1994) 116:11612-11613). Lastly, these enzymes are particularly well-known for generating an impressive range of asymmetric centers in their products in a highly controlled manner. The polyketides and antibiotics produced by the methods of the present invention are typically single stereoisomeric forms. Although the compounds of the invention can occur as mixtures of stereoisomers, it is more practical to generate individual stereoisomers using this system. Thus, the combinatorial potential within modular PKS pathways based on any naturally occurring modular, such as the erythromycin, PKS scaffold is virtually unlimited.

In addition, the nature of the acyl transferase (AT) appears to determine the nature of the extended unit which is added by the module in question. As noted, picromycin module 2 contains an AT which uses malonyl CoA as an extender; the remaining modules utilize methyl malonyl CoA. This results in the absence of a methyl group at C10. By substituting AT activity-encoding regions from various PKS genes, or by mutagenizing the AT unit in a module of a host scaffolding PKS gene, the nature of the extender unit, and thus the nature of R¹-R⁶ may readily be varied.

In general, the polyketide products of the PKS must be further modified, typically by hydroxylation and glycosylation, in order to exhibit antibiotic activity. As described above, hydroxylation results in the novel polyketides of the present invention which contain hydroxyl groups at C6 and/or C12. The presence of hydroxyl groups at these positions is thought to enhance the antibiotic activity. It is clear that glycosylation is important in antibiotic activity as well.

Methods for glycosylating the polyketides are generally known in the art; the glycosylation may be effected intracellularly by providing the appropriate glycosylation enzymes or may be effected in vitro using chemical synthetic means as described in parent application U.S. Ser. No. 09/073,538.

The antibiotic modular polyketides may contain any of a number of different sugars, although D-desosamine, or a close analog thereof, is most common. Erythromycin, picromycin, narbomycin and methymycin contain desosamine. Erythromycin also contains L-cladinose (3-O-methyl mycarose). Tylosin contains mycaminose (4-hydroxy desosamine), mycarose and 6-deoxy-D-allose. 2-acetyl-1-bromodesosamine has been used as a donor to glycosylate polyketides by Masamune et al. J Am Chem Soc (1975) 97:3512, 3513. Other, apparently more stable, donors include glycosyl fluorides, thioglycosides, and trichloroacetimidates; Woodward, R. B. et al. J Am Chem Soc (1981) 103:3215; Martin, S. F. et al. Am Chem Soc (1997) 119:3193; Toshima, K. et al. J Am Chem Soc (1995) 117:3717; Matsumoto, T. et al. Tetrahedron Lett (1988) 5 29:3575. Glycosylation can also be effected using the macrolides as starting materials and using mutants of S. erythraea that are unable to synthesize the macrolides to make the conversion.

In general, the approaches to effecting glycosylation mirror those described above with respect to hydroxylation. The purified enzymes, isolated from native sources or recombinantly produced may be used in vitro. Alternatively, glycosylation may be effected intracellularly using endogenous or recombinantly produced intracellular glycosylases. In addition, synthetic chemical methods may be employed.

Methods to Construct Multiple Modular PKS Derived from a Naturally Occurring PKS

The derivatives of the a naturally occurring PKS can be prepared by manipulation of the relevant genes. A large number of modular PKS gene clusters have been mapped and/or sequenced, including erythromycin, soraphen A, rifamycin, and rapamycin, which have been completely mapped and sequenced, and FK506 and oleandomycin which have been partially sequenced, and candicidin, avermectin, and nemadectin which have been mapped and partially sequenced. Additional modular PKS gene clusters are expected to be available as time progresses. The present invention provides the picromycin PKS. These genes can be manipulated using standard techniques to delete or inactivate activity encoding regions, insert regions of genes encoding corresponding activities form the same or different PKS system, or otherwise mutated using standard procedures for obtaining genetic alterations. Of course, portions of, or all of, the desired derivative coding sequences can be synthesized using standard solid phase synthesis methods such as those described by Jaye et al., J Biol Chem (1984) 259:6331 and which are available commercially from, for example, Applied Biosystems, Inc.

In order to obtain nucleotide sequences encoding a variety of derivatives of the naturally occurring PKS, and thus a variety of polyketides for construction of a library, a desired number of constructs can be obtained by “mixing and matching” enzymatic activity-encoding portions, and mutations can be introduced into the native host PKS gene cluster or portions thereof. Components of the picromycin PKS are made available by the present invention.

Mutations can be made to the native sequences using conventional techniques. The substrates for mutation can be an entire cluster of genes or only one or two of them; the substrate for mutation may also be portions of one or more of these genes. Techniques for mutation include preparing synthetic oligonucleotides including the mutations and inserting the mutated sequence into the gene encoding a PKS subunit using restriction endonuclease digestion. (See, e.g., Kunkel, T. A. Proc Natl Acad Sci USA (1985) 82:448; Geisselsoder et al. Bio Techniques (1987) 5:786.) Alternatively, the mutations can be effected using a mismatched primer (generally 10-20 nucleotides in length) which hybridizes to the native nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. Zoller and Smith, Methods Enzymol (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al Proc Natl Acad Sci USA (1982) 79:6409. PCR mutagenesis will also find use for effecting the desired mutations.

Random mutagenesis of selected portions of the nucleotide sequences encoding enzymatic activities can be accomplished by several different techniques known in the art, e.g., by inserting an oligonucleotide linker randomly into a plasmid, by irradiation with X-rays or ultraviolet light, by incorporating incorrect nucleotides during in vitro DNA synthesis, by error-prone PCR mutagenesis, by preparing synthetic mutants or by damaging plasmid DNA in vitro with chemicals. Chemical mutagens include, for example, sodium bisulfite, nitrous acid, nitrosoguanidine, hydroxylamine, agents which damage or remove bases thereby preventing normal base-pairing such as hydrazine or formic acid, analogues of nucleotide precursors such as 5-bromouracil, 2-aminopurine, or acridine intercalating agents such as proflavine, acriflavine, quinacrine, and the like.

Generally, plasmid DNA or DNA fragments are treated with chemicals, transformed into E. coli and propagated as a pool or library of mutant plasmids.

In addition to providing mutated forms of regions encoding enzymatic activity, regions encoding corresponding activities from different PKS synthases or from different locations in the same PKS synthase can be recovered, for example, using PCR techniques with appropriate primers. By “corresponding” activity encoding regions is meant those regions encoding the same general type of activity—e.g., a ketoreductase activity in one location of a gene cluster would “correspond” to a ketoreductase-encoding activity in another location in the gene cluster or in a different gene cluster; similarly, a complete reductase cycle could be considered corresponding—e.g., KR/DH/ER would correspond to KR alone.

If replacement of a particular target region in a host polyketide synthase is to be made, this replacement can be conducted in vitro using suitable restriction enzymes or can be effected in vivo using recombinant techniques involving homologous sequences framing the replacement gene in a donor plasmid and a receptor region in a recipient plasmid. Such systems, advantageously involving plasmids of differing temperature sensitivities are described, for example, in PCT application WO 96/40968.

The vectors used to perform the various operations to replace the enzymatic activity in the host PKS genes or to support mutations in these regions of the host PKS genes may be chosen to contain control sequences operably linked to the resulting coding sequences in a manner that expression of the coding sequences may be effected in a appropriate host. However, simple cloning vectors may be used as well.

If the cloning vectors employed to obtain PKS genes encoding derived PKS lack control sequences for expression operably linked to the encoding nucleotide sequences, the nucleotide sequences are inserted into appropriate expression vectors. This need not be done individually, but a pool of isolated encoding nucleotide sequences can be inserted into host vectors, the resulting vectors transformed or transfected into host cells and the resulting cells plated out into individual colonies.

Suitable control sequences include those which function in eucaryotic and procaryotic host cells. Preferred host include fungal systems such as yeast and procaryotic hosts, but single cell cultures of, for example, mammalian cells could also be used. There is no particular advantage, however, in using such systems. Particularly preferred are yeast and procaryotic hosts which use control sequences compatible with Streptomyces spp. Suitable controls sequences for single cell cultures of various types of organisms are well known in the art. Control systems for expression in yeast, including controls which effect secretion are widely available are routinely used. Control elements include promoters, optionally containing operator sequences, and other elements depending on the nature of the host, such as ribosome binding sites. Particularly useful promoters for procaryotic hosts include those from PKS gene clusters which result in the production of polyketides as secondary metabolites, including those from aromatic (Type II) PKS gene clusters. Examples are act promoters, tcm promoters, spiramycin promoters, and the like. However, other bacterial promoters, such as those derived from sugar metabolizing enzymes, such as galactose, lactose (lac) and maltose, are also useful. Additional examples include promoters derived from biosynthetic enzymes such as tryptophan (trp), the β-lactamase (bla), bacteriophage lambda PL, and T5. In addition, synthetic promoters, such as the tac promoter (U.S. Pat. No. 4,551,433), can be used.

Other regulatory sequences may also be desirable which allow for regulation of expression of the PKS replacement sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

Selectable markers can also be included in the recombinant expression vectors. A variety of markers are known which are useful in selecting for transformed cell lines and generally comprise a gene whose expression confers a selectable phenotype on transformed cells when the cells are grown in an appropriate selective medium. Such markers include, for example, genes which confer antibiotic resistance or sensitivity to the plasmid. Alternatively, several polyketides are naturally colored and this characteristic provides a built-in marker for screening cells successfully transformed by the present constructs.

The various PKS nucleotide sequences, or a cocktail of such sequences, can be cloned into one or more recombinant vectors as individual cassettes, with separate control elements, or under the control of, e.g., a single promoter. The PKS subunits or cocktail components can include flanking restriction sites to allow for the easy deletion and insertion of other PKS subunits or cocktail components so that hybrid PKSs can be generated. The design of such unique restriction sites is known to those of skill in the art and can be accomplished using the techniques described above, such as site-directed mutagenesis and PCR.

As described above, particularly useful control sequences are those which themselves, or using suitable regulatory systems, activate expression during transition from growth to stationary phase in the vegetative mycelium. The system contained in the illustrative plasmid pRM5, i.e., the actI/actIII promoter pair and the actII-ORF4, an activator gene, is particularly preferred. Particularly preferred hosts are those which lack their own means for producing polyketides so that a cleaner result is obtained. Illustrative host cells of this type include the modified S. coelicolor CH999 culture described in PCT application WO 96/40968 and similar strains of S. lividans.

The expression vectors containing nucleotide sequences encoding a variety of PKS systems for the production of different polyketides are then transformed into the appropriate host cells to construct the library. In one straightforward approach, a mixture of such vectors is transformed into the selected host cells and the resulting cells plated into individual colonies and selected for successful transformants. Each individual colony will then represent a colony with the ability to produce a particular PKS synthase and ultimately a particular polyketide. Typically, there will be duplications in some of the colonies; the subset of the transformed colonies that contains a different PKS in each member colony can be considered the library. Alternatively, the expression vectors can be used individually to transform hosts, which transformed hosts are then assembled into a library. A variety of strategies might be devised to obtain a multiplicity of colonies each containing a PKS gene cluster derived from the naturally occurring host gene cluster so that each colony in the library produces a different PKS and ultimately a different polyketide. The number of different polyketides that are produced by the library is typically at least four, more typically at least ten, and preferably at least 20, more preferably at least 50, reflecting similar numbers of different altered PKS gene clusters and PKS gene products. The number of members in the library is arbitrarily chosen; however, the degrees of freedom outlined above with respect to the variation of starter, extender units, stereochemistry, oxidation state, and chain length is quite large.

Methods for introducing the recombinant vectors of the present invention into suitable hosts are known to those of skill in the art and typically include the use of CaCl₂ or other agents, such as divalent cations, lipofection, DMSO, protoplast transformation and electroporation.

As disclosed in copending Application Ser. No. 08/989,332 filed Dec. 11, 1997, incorporated herein by reference, a wide variety of hosts can be used, even though some hosts natively do not contain the appropriate post-translational mechanisms to activate the acyl carrier proteins of the synthases. These hosts can be modified with the appropriate recombinant enzymes to effect these modifications.

The polyketide producing colonies can be identified and isolated using known techniques and the produced polyketides further characterized. The polyketides produced by these colonies can be used collectively in a panel to represent a library or may be assessed individually for activity.

The libraries can thus be considered at four levels: (1) a multiplicity of colonies each with a different PKS encoding sequence encoding a different PKS cluster but all derived from a naturally occurring PKS cluster; (2) colonies which contain the proteins that are members of the PKS produced by the coding sequences; (3) the polyketides produced; and (4) antibiotics derived from the polyketides. Of course, combination libraries can also be constructed wherein members of a library derived, for example, from the erythromycin PKS can be considered as a part of the same library as those derived from, for example, the rapamycin PKS cluster.

Colonies in the library are induced to produce the relevant synthases and thus to produce the relevant polyketides to obtain a library of candidate polyketides. The polyketides secreted into the media can be screened for binding to desired targets, such as receptors, signaling proteins, and the like. The supernatants per se can be used for screening, or partial or complete purification of the polyketides can first be effected. Typically, such screening methods involve detecting the binding of each member of the library to receptor or other target ligand. Binding can be detected either directly or through a competition assay. Means to screen such libraries for binding are well known in the art.

Alternatively, individual polyketide members of the library can be tested against a desired target. In this event, screens wherein the biological response of the target is measured can more readily be included.

The parent application herein describes the preparation of a large number of polyketides. These polyketides are useful intermediates in formation of compounds with antibiotic activity through hydroxylation and glycosylation reactions as described above. As indicated above, the individual polyketides are reacted with suitable sugar derivatives to obtain compounds of antibiotic activity. Antibiotic activity can be verified using typical screening assays such as those set forth in Lehrer, R. et al. J Immunol Meth (1991) 137:167-173.

New polyketides which are the subject of the invention are hydroxylated forms of those described in the parent application.

New antibiotics which are the subject of the invention include the hydroxylated and glycosylated forms of the polyketides described in the parent application..

The compounds of the present invention are thus optionally glycosylated forms of the polyketide set forth in formula (2) below which are hydroxylated at either the 6-carbon or the 12-carbon or both. The compounds of formula (2) can be prepared using six modules of a modular polyketide synthase, modified or prepared in hybrid form as herein described. These polyketides have the formula

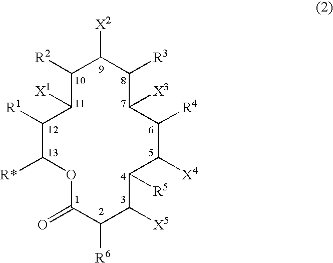

including the glycosylated and isolated stereoisomeric forms thereof;

wherein R* is a straight chain, branched or cyclic, saturated or unsaturated substituted or unsubstituted hydrocarbyl of 1-15C;

each of R¹-R⁶ is independently H or alkyl (1-4C) wherein any alkyl at R¹ may optionally be substituted;

each of X¹-X⁵ is independently H₂, HOH or ═O; or

each of X¹-X⁵ is independently H and the compound of formula (5) contains a π-bond in the ring adjacent to the position of said X at 2-3, 4-5, 6-7, 8-9 and/or 10-11;

with the proviso that:

at least two of R¹-R⁶ are alkyl (1-4C).

Preferred compounds comprising formula 2 are those wherein at least three of R¹-R⁵ are alkyl (1-4C), preferably methyl or ethyl; more preferably wherein at least four of R¹-R⁵ are alkyl (1-4C), preferably methyl or ethyl.

Also preferred are those wherein X² is H₂, ═O or H [symbol] . . . OH, and/or X³ is H, and/or X¹ is OH and/or X⁴ is OH and/or X⁵ is OH.

Also preferred are compounds with variable R* when R¹-R⁵ are methyl, X² is ═O, and X¹, X⁴ and X⁵ are OH. The glycosylated forms of the foreoging are also preferred.

The following examples are intended to illustrate, but not to limit the invention.

### Materials and Methods

### General Techniques

Bacterial strains, plasmids, and culture conditions. S. coelicolor CH999 described in WO 95/08548, published Mar. 30, 1995 was used as an expression host. DNA manipulations were performed in Escherichia coli MC1061. Plasmids were passaged through E. coli ET12567 (dam dcm hsdS Cm^{r}) (MacNeil, D. J. J Bacteriol (1988) 170:5607) to generate unmethylated DNA prior to transformation of S. coelicolor. E. coli strains were grown under standard conditions. S. coelicolor strains were grown on R2YE agar plates (Hopwood, D. A. et al. Genetic manipulation of Streptomyces. A laboratory manual. The John Innes Foundation: Norwich, 1985). pRM5, also described in WO 95/08548, includes a colEI replicon, an appropriately truncated SCP2*Streptomyces replicon, two act-promoters to allow for bidirectional cloning, the gene encoding the actII-ORF4 activator which induces transcription from act promoters during the transition from growth phase to stationary phase, and appropriate marker genes. Engineered restriction sites facilitate the combinatorial construction of PKS gene clusters starting from cassettes encoding individual domains of naturally occurring PKSs.

When pRM5 is used for expression of PKS, (i) all relevant biosynthetic genes are plasmid-borne and therefore amenable to facile manipulation and mutagenesis in E. coli, (ii) the entire library of PKS gene clusters can be expressed in the same bacterial host which is genetically and physiologically well-characterized and presumably contains most, if not all, ancillary activities required for in vivo production of polyketides, (iii) polyketides are produced in a secondary metabolite-like manner, thereby alleviating the toxic effects of synthesizing potentially bioactive compounds in vivo, and (iv) molecules thus produced undergo fewer side reactions than if the same pathways were expressed in wild-type organisms or blocked mutants.

Manipulation of DNA and organisms. Polymerase chain reaction (PCR) was performed using Taq polymerase (Perkin Elmer Cetus) under conditions recommended by the enzyme manufacturer. Standard in vitro techniques were used for DNA manipulations (Sambrook, et al. Molecular Cloning: A Laboratory Manual (Current Edition)). E. coli was transformed with a Bio-Rad E. coli Pulsing apparatus using protocols provided by Bio-Rad. S. coelicolor was transformed by standard procedures (Hopwood, D. A. et al. Genetic manipulation of Streptomyces. A laboratory manual. The John Innes Foundation: Norwich, 1985) and transformants were selected using 2 mL of a 500 μg/ml thiostrepton overlay.

### EXAMPLE 1

### Construction of the Complete Picromycin PKS

Cosmid pKOS023-27 was isolated from a genomic library of S. venezuelae. The structure of pKOS023-27 is shown in FIG. 5 and confirms that this contains the complete set of open reading frames corresponding to the picromycin PKS.

The identity of the sequences in this cosmid with those encoding the picromycin PKS was confirmed by using the 2.4 kb EcoRI/KpnI fragment and the 2.1 kb KpnI/Xho1 fragment isolated from the cosmid ligated together and cloned into pLitmus 28 to give pKOS039-07. The 4.5 kb HindIII/SpeI fragment from this plasmid was cloned into the 2.5 kb HindIII/NheI fragment of pSet 152 which contains the E. coli origins for replication and an apramycin-resistant gene to obtain pKOS039-16. This vector was used to transform S. venezuelae to apramycin-resistance. The transformed S. venezuelae lost its ability to produce picromycin indicating that the plasmid was integrated into the appropriate location on the chromosome. Either loss of the integrated vector or introduction of the picA gene on pWHM3 under the control of the ermE* on plasmid pKOS039-27 were able to restore picromycin synthesis, although at a lower level.

### EXAMPLE 2

### Cloning of picK, the Narbomycin 12-Hydroxylase Gene from S. venezuelae

Genomic DNA isolated from Streptomyces venezuelae ATCC15439 using standard procedures (100 μg) was partially digested with Sau3AI endonuclease to generate fragments ca. 40-kbp in length. SuperCosI (Stratagene) DNA cosmid arms were prepared as directed by the manufacturer. A cosmid library was prepared by ligating 2.5 μg of the digested genomic DNA with 1.5 μg of cosmid arms in a 20 μL reaction. One microliter of the ligation mixture was propagated in E. coli XL1-Blue MR (Stratagene) using a GigapackIII XL packaging extract kit (Stratagene). The resulting library of ca. 3000 colonies was plated on a 10×150 mm agar plate and replicated to a nylon membrane.

The library was initially screened by direct colony hybridization with a DNA probe specific for ketosynthase domains of polyketide synthases. Colonies were alkaline lysed, and the DNA was crosslinked to the membrane using UV irradiation. After overnight incubation with the probe at 42° C., the membrane was washed twice at 25° C. in 2×SSC buffer+0.1% SDS for 15 minutes, followed by two 15 minutes washes with 2×SSC buffer at 55° C. Approximately 30 colonies gave positive hybridization signals. Several candidate cosmids were selected and divided into two classes based on restriction digestion patterns. A representative cosmid was selected from each class for further analysis.

Each cosmid was probed by Southern hybridization using a labeled DNA fragment amplified by PCR from the Saccharopolyspora erythraea 12-hydroxylase gene, eryK. The cosmids were digested with BamHI endonuclease and electrophoresed on a 1% agarose gel, and the resulting fragments were transferred to a nylon membrane. The membrane was incubated with the eryK probe overnight at 42° C., washed twice at 25° C. in 2×SSC buffer+0.1% SDS for 15 minutes, followed by two 15 minutes washes with 2×SSC buffer at 50° C. One cosmid, pKOS023-26, produced a 3.0-kbp fragment which hybridized with the probe under these conditions. This fragment was subcloned into the PCRscript (Stratagene) cloning vector to yield plasmid pKOS023-28, and sequenced. A ca. 1.2-kbp gene, designated picK, was found having homology to eryK and other known macrolike cytochrome P450 hydroxylases.

The complete sequence of the open reading frame and the deduced amino acid sequence are shown in FIG. 6, pages 4-5 (nucleotide sequence nt 1356-2606) and page 7 (amino acid sequence).

In addition, the glycosylase was retrieved on the cosmid KOS023-26 and the open reading frame and deduced amino acid sequence are shown in FIG. 5, page 13 (nucleotide sequence, nt 36159-37439) and page 23 (amino acid sequence).

### EXAMPLE 3

### Construction of picK Expression Plasmids for E. coli

A. The picK Gene was PCR Amplified Using Oligonucleotide Primers

(forward
5′-TTGCATGCATATGCGCCGTACCCAGCAGGGAACGACC;
reverse
5′-TTGAATTCTCAACTAGTACGGCGGCCCGCCTCCCGTCC).

These primers alter the Streptomyces GTG start codon to ATG and introduce a SpeI site at the C-terminal end of the gene, resulting in the substitution of a serine for the terminal glycine amino acid residue. Following subcloning of the PCR product, the 1.3 kb gene fragment was cloned into the NdeI/XhoI sites of the T7 expression vector pET22b (Novagen, Madison, Wis.) to generate pKOS023-61. A short linker fragment encoding 6 histidine residues and a stop codon was introduced into the SpeI site to obtain pKOS023-68.

Alternatively, the PCR product was cloned into the SrfI site of PCRscript (Stratagene) to generate pKOS023-60. This plasmid was digested with NdeI/XhoI and the resulting 1.3 kb fragment ligated with correspondingly restricted pET22V vector (Invitrogen) to obtain pKOS023-61.

### EXAMPLE 4

### Hydroxylation of Narbomycin by Narbomycin 12-Hydroxylase

Narbomycin was converted to picromycin with a crude cell-free extract from E. coli expressing picK. Narbomycin was purified from a culture of S. narbonensis, and upon LC/MS analysis gave a single peak of [M+H]⁺=510. Plasmid pKOS023-61 (See Example 3) was transformed into E. coli BL21-DE3. Successful transformants were grown in LB-containing carbenicillin (100 μg/ml) at 37° C. to an OD₆₀₀ of 0.6. Isopropyl-b-D-thiogalactopyranoside (IPTG) was added to a final concentration of 1 mM and the cells were grown for an additional 3 hours before harvesting. The cells were collected by centrifugation and frozen at −80° C. A control culture of BL21-DE3 containing the vector plasmid pET21c (Invitrogen) was prepared in parallel.

The frozen BL21-DE3/pKOS023-61 cells were thawed, suspended in 2 μL of cold cell disruption buffer (5 mM imidazole, 500 mM NaCl, 20 mM Tris/HCl, pH 8.0) and sonicated to facilitate lysis. Cellular debris and supernatant were separated by centrifugation and subjected to SDS-PAGE on 10-15% gradient gels, with Coomassie Blue staining, using a Pharmacia Phast Gel Electrophoresis system. the soluble crude extract from BL21-DE3/pKOS023-61 contained a Coomassie stained band of Mᵣ˜46 kDa which was absent in the control strain BL21-DE3/pET21c.

The hydroxylase activity of the picK protein was assayed as follows. The crude supernatant (20 μl) was added to a reaction mixture (100 μl total volume) containing 50 mM Tris/HCl (pH 7.5), 20 μM spinach ferredoxin, 0.025 Unit of spinach ferredoxin:NADP⁺ oxidoreductase, 0.8 Unit of glucose-6-phosphate dehydrogenase, 1.4 mM NADP⁺, 7.6 mM glucose-6phosphate, and 20 nmol of narbomycin. The reaction was allowed to proceed for 105 minutes at 30° C. Half of the reaction mixture was loaded onto an HPLC, ,and the effluent was analyzed by evaporative light scattering (ELSD) and mass spectrometry. The control extract (BL21-DE3/pET21c) was processed identically. The BL21-DE3/pKOS023-61 reaction contained a compound not present in the control having the same retention time, molecular weight and mass fragmentation pattern as picromycin ([M+H]⁺=526). The conversion of narbormycin to picromycin under these conditions was estimated to be greater than 90% by ELSD peak area.

### EXAMPLE 5

### Preparation of Cell Extracts and Purification of PicK/6-His

To produce His-tailed hydroxylase, pKOS023-68, described in Example 3, was transfected into E. coli BL21 (DE3) and cultured as described in Example 4. The cells were harvested and the picK protein purified.

All purification steps were performed at 4° C. E. coli cell pellets were suspended in 32 μL of cold binding buffer (20 mM Tris/HCl, pH 8.0, 5 mM imidazole, 500 mM NaCl) per mL of culture and lysed by sonication. for analysis of E. coli cell-free extracts, the cellular debris was removed by low-speed centrifugation and the supernatant was used directly in assays. For purification of PicK/6-His, the supernatant was loaded (0.5 mL/min.) onto a 5 mL HiTrap Chelating column (Pharmacia, Piscataway, N.J.), equilibrated with binding buffer. The column was washed with 25 μL of binding buffer and the protein was eluted with a 35 μL linear gradient (5-500 mM imidazole in binding buffer). Column effluent was monitored at 280 nm and 416 nm. Fractions corresponding to the 416 nm absorbance peak were pooled and dialyzed against storage buffer (45 mM Tris/HCl, pH 7.5, 0.1 mM EDTA, 0.2 mM DTT, 10% glycerol). The purified 46 kDa protein was analyzed by SDS-PAGE using coomassie blue staining, and enzyme concentration and yield were determined.

### EXAMPLE 6

### 6-Hydroxylation of 3.6-Dideoxy-3-Oxoerythronolide B using the eryF Hydroxylase

The 6-hydroxylase encoded by eryF was expressed in E. coli, and partially purified.

The hydroxylase (100 pmol in 10 μL) was added to a reaction mixture (100 μl total volume) containing 50 mM Tris/HCl (pH 7.5), 20 μM spinach ferredoxin, 0.025 Unit of spinach ferredoxin:NADP⁺ oxidoreductase, 0.8 Unit of glucose-6-phosphate dehydrogenase, 1.4 mM NADP⁺, 7.6 mM glucose-6-phosphate, and 10 nmol 6-deoxyerythronolide B. The reaction was allowed to proceed for 90 minutes at 30° C. Half of the reaction mixture was loaded onto an HPLC, and the effluent was analyzed by mass spectrometry. This revealed production of erythronolide B as evidenced by a new peak eluting earlier in the gradient and showing [M+H]⁺=401. Conversion was estimated at 50% based on relative total ion counts.

### EXAMPLE 7

### Kinetic Assays with Narbomycin

Narbomycin was purified from a culture of Streptomyces narbonensis ATCC19790. reactions for kinetic assays (100 μL) consisted of 50 mM Tris/HCl (pH 7.5), 100 μM spinach ferredoxin, 0.025 Unit of spinach ferredoxin:NADP⁺ oxidoreductase,, 0.8 U glucose-6-phosphate dehydrogenase, 1.4 mM NADP⁺, 7.6 mM glucose-6-phosphate, 20-500 μM narbomycin substrate, and 50-500 nM of picK. The reaction proceeded at 30° C. and samples were withdrawn for analysis at 5, 10, 15, and 90 minutes. Reactions were stopped by heating to 100° C. for 1 minute and denatured protein was removed by centrifugation. Depletion of narbomycin and formation of picromycin were determined by high performance liquid chromatography (HPLC, Beckman C-18 0.46×15 cm column) coupled to stmospheric pressure chemical ionization (APCI) mass spectroscopic detection (Perkin Elmer/Sciex API 100) and evaporative light scattering detection (Alltech 500 ELSD).

### EXAMPLE 8

### Measurement of Antibacterial Activity

Antibacterial activity was determined using either disk diffusion assays with Bacillus cereus as the test organism or by measurement of minimum inhibitory concentrations (MIC) in liquid culture against sensitive and resistant strains of Staphylococcus pneumoniae.

### EXAMPLE 9

### Expression of the picK Gene Encoding the Hydroxylase in S. narbonensis

In order to improve the yield and purity of picromycin produced in S. narbonensis, the picK gene was expressed in this host.

The picK gene was amplified from pKOS023-26 using the primers:

| | |
|---|---|
| N3903: | 5′-TCCTCTAGACGTTTCCGT-3′ |
| N3904: | 5′-TGAAGCTTGAATTCAACCGGT-3′ |

to obtain a 1.29 kb product. The product was treated with XbaI/HindIII and cloned into similarly treated with pWHM1104 to provide pKOS039-01 placing the gene under the ermE* promoter. The resulting plasmid was transformed into purified stocks of S. narbonensis by protoplast fusion and electroporation. The transformants were grown in suitable media and shown to convert narbomycin to picromycin at a yield of over 95%.

### EXAMPLE 10

### Expression of Desosaminyl Transferase into S. erythraea

To provide S. erythraea with suitable additional enzymes for glycosylation, the picG gene (desosaminyl transferase) was amplified from pKOS023-27 using the primers:

| | |
|---|---|
| N3917: | 5′-CCCTGCAGCGGCAAGGAAGGACACGACGCCA-3′ |
| N3918: | 5′-AGGTCTAGAGCTCAGTGCCGGGCGTCGGCCGG-3′ |

to give a 1.5 kb product which was treated with PstI/XbaI and ligated into similarly treated pKOS039-06 along with the PstI/HindIII fragment of pWHM1104 to provide pKOS039-14 placing the picG gene after DEBS module 2 and under the control of the ermE* promoter. The vector was then transformed into S. erythraea by treating the protoplast with the plasmid.

### EXAMPLE 11

### Construction of Hybrid Erythromycin/Picromycin PKS

Table 1 shows a summary of constructs which are hybrids of portions of the picromycin PKS and portions of rapamycin and/or erythromycin PKS. In the first constructs, pKOS039-18 and pKOS039-19, the picromycin module 6 ACP and thioesterase replaced the corresponding region as well as the KR in the erythromycin cluster; in pKOS039-19 the erythromycin cluster further contains a KS1 knock-out—i.e., the ketosynthase in module 1 was disabled. The KS1 knock-out is described in detail in PCT application US/96/11317, the disclosure of which is incorporated herein by reference. To construct pKOS039-18, the 2.33 kb BamHI/EcoRI fragment of pKOS023-27 which contains the desired sequence was subcloned on pUC19 and used as the template for PCR. The primers were

| | |
|---|---|
| N3905: | 5′-TTTATGCATCCCGCGGGTCCCGGCGAG3′ |
| N3906: | 5′-TCAGAATTCTGTCGGTCACTTGCCCGC3′ |

The 1.6 kb PCR product was digested with PstI/EcoRI and cloned into the corresponding sites of pKOS015-52 and pLitmus 28 to provide pKOS039-12 and pKOS039-13, respectively. The BgIII/EcoRI fragment of pKOS039-12 was cloned into pKOS011-77 which contains wild-type erythromycin gene cluster and into JRJ2 which corresponds to this plasmid that contains the KSI knock-out. pKOS039-18 and pKOS039-19, respectively, were obtained.

These two plasmids were transfected into S. coelicolor CH999 by protoplast fusion.

The resulting cells were cultured under conditions whereby expression was obtained and the expected polyketides were obtained from this culture. From pKOS039-18, the product was 3-keto-6 dEB. From pKOS039-19, when activated isobutyrate was used as the starting material, propyl-3-keto-6 dEB was obtained.

Table 1 shows additional constructs and the nature of the expected product.

When CH999 is used as a host, the product is the unconverted polyketide; when cultured in strain K39-03, which contains the required hydroxylase and glycosylation enzymes, the corresponding antibiotics were obtained.

TABLE 1

| # | Substrate | 1 | 2 | 3 | 4 | 5 | 6 | Host | Product |
|---|---|---|---|---|---|---|---|---|---|
| 1 | — | ery | ery | ery | ery | ery | ery | CH999 | 3-keto-6-dEB |
| | | | | | | | KR-ACP-TE → | | |
| | | | | | | | pic-ACP-TE | | |
| 2 | butyrate | ery | ery | ery | ery | ery | ery | CH999 | propyl-3-keto-6-dEB |
| | | KSI* | | | | | KR-ACP-TE → | | |
| | | | | | | | pic-ACP-TE | | |
| 3 | — | pic | pic | ery | ery | ery | ery | CH999 | 10-methyl narbonolide |
| | | | AT → ery AT | | | | KR-ACP-TE → | | |
| | | | | | | | pic-ACP-TE | | |
| 4 | butyrate | pic | pic | ery | ery | ery | ery | CH999 | propyl-10-methyl |
| | | KSI* | AT → ery AT | | | | KR-ACP-TE → | | narbonolide |
| | | | | | | | pic-ACP-TE | | |
| 5 | — | ery | ery | ery | ery | ery | ery | CH999 | 10-methyl narbonolide |
| | | | KR → rap | | | | KR-ACP-TE → | | |
| | | | DH/KR | | | | pic-ACP-TE | | |
| 6 | butyrate | ery | ery | ery | ery | ery | ery | CH999 | propyl-10-methyl |
| | | KSI* | KR → rap | | | | KR-ACP-TE → | | narbonolide |
| | | | DH/KR | | | | pic-ACP-TE | | |
| 7 | butyrate | pic | pic | ery | ery | ery | ery | CH999 | propyl-10, 11-dehydro |
| | | KSI* | AT → ery AT | | | | | | 6dEB |
| 8 | butyrate | pic | pic | pic | pic | pic | pic | K3903 | propyl-10-methyl |
| | | KSI* | AT → ery AT | | | | | | picromycin |
| 9 | — | pic | pic | pic | pic | pic | pic | K3903 | 10-methyl picromycin |
| | | | AT → ery AT | | | | | | |
| 10 | — | ery | ery | ery | ery | ery | ery | K3903 | 5-sugar-3-keto-6-dEB |
| | | | | | | | KR-ACP-TE → | | |
| | | | | | | | pic ACP-TE | | |

In Table 1 “ery” refers to the numbered module from the erythromycin PKS; “pic” refers to the relevant module on the picromycin PKS. The notations under the designations indicate any alterations that were made in the module. Thus, embodiment #1 is that described hereinabove where the KR-ACP-TE of module 6 of erythromycin was replaced by the ACP-TE corresponding portion of module 6 of the picromycin PKS. The CH999 host does not glycosylate the corresponding ketolides, but K39-03 has this ability. When module 1 has a KS1 knock-out (symbolized KS1*) butyrate was supplied as the substrate, leading to the corresponding ketolide or antibiotic with a propyl chain at carbon 13.

1. An isolated nucleic acid which comprises a nucleotide sequence encoding at least one activity of a picromycin PKS.

2. The isolated nucleic acid of claim 1 which comprises the nucleotide sequence encoding at least one module of the picromycin PKS.

3. The isolated nucleic acid of claim 2 which comprises the nucleotide sequence encoding the protein encoded by at least one open reading frame of the picromycin PKS.

4. An isolated nucleic acid which comprises a nucleotide sequence encoding picK, the narbomycin 12-hydroxylase gene, or which comprises a nucleotide sequence encoding the desosaminyl transferase gene from S. venezuelae.

5. A recombinant nucleic acid molecule which comprises a first nucleotide sequence encoding at least one activity of the picromycin PKS operably linked to at least one second nucleotide sequence that effects the expression of said first nucleotide sequence in a recombinant host.

6. The recombinant nucleic acid molecule of claim 5 wherein the first nucleotide sequence encodes at least one module of the picromycin PKS.

7. The recombinant nucleic acid molecule of claim 5 wherein the first nucleotide sequence encodes the protein encoded by at least one open reading frame of the picromycin PKS.

8. The nucleic acid molecule of claim 5 wherein said second nucleotide sequence is compatible with yeast, E. coli or Streptomyces host cells.

9. The nucleic acid molecule of claim 6 wherein said second nucleotide sequence is compatible with yeast, E. coli or Streptomyces host cells.

10. The nucleic acid molecule of claim 7 wherein said second nucleotide sequence is compatible with yeast, E. coli or Streptomyces host cells.

11. Recombinant host cells containing the recombinant nucleic acid molecule of claim 5.

12. Recombinant host cells containing the recombinant nucleic acid molecule of claim 6.

13. Recombinant host cells containing the recombinant nucleic acid molecule of claim 7.

14. A method to produce a protein having the activity associated with a conversion step in a PKS pathway which method comprises culturing the cells of claim 12 under conditions wherein a protein having such activity is produced.

15. A method to effect a conversion representing a step in the synthesis of a polyketide which method comprises providing the starting material for said conversion to the cells of claim 12.

16. A protein having PKS activity produced by the method of claim 14.

17. A method to effect a conversion of the PKS pathway which comprises contacting a starting material for said conversion with the protein of claim 16.

18. A method to prepare a nucleic acid with the nucleotide sequence encoding a modified PKS from a nucleotide sequence encoding the picromycin PKS wherein said picromycin PKS contains first regions that encode enzymatic activities and second regions which encode scaffolding amino acid sequences, which method comprises modifying at least one said first region.

19. The method of claim 18 wherein said modifying comprises deleting or inactivating at least one said first region; or
wherein said modifying comprises replacing at least one said first region with a region encoding the corresponding enzymatic activity from a different naturally occurring PKS gene or from a different region of the picromycin PKS.

20. A nucleic acid molecule comprising a nucleotide sequence encoding a modified PKS obtainable by the method of claim 18.

21. A recombinant nucleic acid molecule which comprises a first nucleotide sequence encoding a modified PKS obtainable by the method of claim 18 operably linked to at least one second nucleotide sequence that effects the expression of said first nucleotide sequence in a recombinant host.

22. A host cell modified to contain the recombinant nucleic acid molecule of claim 21.

23. A method to prepare a functional polyketide synthase which method comprises culturing the cells of claim 22 under conditions wherein said polyketide synthase is produced.

24. A polyketide synthase produced by the method of claim 23.

25. A method to prepare a polyketide which method comprises culturing the cells of claim 22 under conditions wherein said polyketide is produced.

26. A novel polyketide prepared by the method of claim 25.

27. A method to prepare an antibiotic which method comprises glycosylating the polyketide of claim 26.

28. An antibiotic prepared by the method of claim 27.

29. The method of claim 18 wherein the first region is the acetyl transferase (AT) of picromycin module 2 and wherein said first region is replaced by eryAT2.

30. The recombinant nucleic acid molecule of claim 21 wherein the first region is the acetyl transferase (AT) of picromycin module 2 and wherein said first region is replaced by eryAT2.

31. A hybrid PKS encoding nucleic acid molecule which comprises a portion of the erythromycin PKS and a portion of the picromycin PKS.

32. The hybrid modular polyketide synthase encoding nucleic acid molecule of claim 31 wherein the acyl carrier protein (ACP) and thioesterase (TE) region of module 6 of the erythromycin PKS is replaced by the corresponding portion of the picromycin PKS.

33. A recombinant nucleic acid molecule which comprises a first nucleotide sequence encoding picK, the narbomycin 12-hydroxylase gene, operably linked to at least one second nucleotide sequence that effects the expression of said first nucleotide sequence in a recombinant host.

34. Recombinant host cells containing the nucleic acid molecule of claim 33.

35. A method to produce narbomycin 12-hydroxylase which method comprises
culturing the cells of claim 34 under conditions wherein said first nucleotide sequence is expressed.

36. Narbomycin 12-hydroxylase prepared by the method of claim 35.

37. A method to obtain a polyketide hydroxylated at position 12 which method comprises treating a precursor lacking hydroxylation at position 12 with the protein of claim 36.

38. A method to obtain a polyketide hydroxylated at position 12 which method comprises culturing S. venezuelae cells modified to delete the picromycin PKS and to contain a substitute PKS for production of a precursor lacking hydroxylation at position 12.

39. A novel polyketide of the formula: 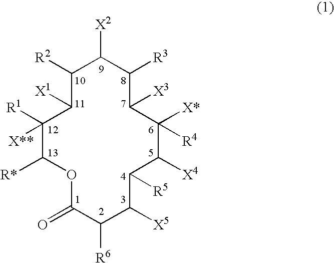
including the glycosylated and isolated stereoisomeric forms thereof,
wherein R* is a straight-chain, branched or cyclic saturated or unsaturated substituted or unsubstituted hydrocarbyl of 1-15C;
each of R¹-R⁶ is independently H or alkyl (¹-4C);
each of X¹-X⁵ is independently H₂, HOH or ═O; or
each of X¹-X⁵ is independently H and the compound of formula 5 contains a π-bond in the ring adjacent to the position of said X at 2-3, 4-5, 6-7, 8-9 and/or 10-11; and
wherein at least one of X* and X** is OH; and
wherein at least two of R¹-R⁶ are alkyl.
